(19) United States
(12) Patent Application Publication
(10) Pub. No.: US 2015/0209416 A1
(43) Pub. Date: Jul. 30, 2015

(54) COMPOSITION AND METHOD FOR CO-ADMINISTRATION WITH A GROWTH PROMOTANT

(71) Applicants: Steven B. Puntenney, Ione, OR (US); Neil E. Forsberg, Corvallis, OR (US); James D. Chapman, Macon, GA (US); Kevin DeHaan, Taylor, MO (US); Troy Wistuba, Ballwin, MO (US); Derek McLean, Corvallis, OR (US); David Calabotta, Quincy, IL (US); Shelby Armstrong, Philomath, OR (US); Jennifer Branson, Corvallis, OR (US)

(72) Inventors: Steven B. Puntenney, Ione, OR (US); Neil E. Forsberg, Corvallis, OR (US); James D. Chapman, Macon, GA (US); Kevin DeHaan, Taylor, MO (US); Troy Wistuba, Ballwin, MO (US); Derek McLean, Corvallis, OR (US); David Calabotta, Quincy, IL (US); Shelby Armstrong, Philomath, OR (US); Jennifer Branson, Corvallis, OR (US)

(73) Assignee: OmniGen Resarch, LLC, Corvallis, OR (US)

(21) Appl. No.: 14/606,862

(22) Filed: Jan. 27, 2015

Related U.S. Application Data
(60) Provisional application No. 61/932,146, filed on Jan. 27, 2014.
(60) Provisional application No. 61/932,149, filed on Jan. 27, 2014.
(60) Provisional application No. 61/932,158, filed on Jan. 27, 2014.
(60) Provisional application No. 61/939,206, filed on Feb. 12, 2014.

(51) Int. Cl. A61K 38/47; A61K 33/06; A61K 31/702; A61K 31/716; A23K 1/16; A61K 31/424; A61K 31/57; A61K 31/55; A23K 1/165; A23K 1/175; A61K 33/00; A61K 31/351

(52) U.S. Cl. CPC A61K 38/47; A61K 33/00; A61K 33/06; A61K 31/702; A61K 31/716; A61K 31/351; A61K 31/424; A61K 31/57; A61K 31/55; A23K 1/165; A23K 1/175; A23K 1/1631; C12Y 302/01006

(57) ABSTRACT

Embodiments of a composition comprising silica, mineral clay, mannans, or any combination thereof are disclosed. The composition may further comprise glucan. The composition is administered to an animal that will be administered, or has been administered, a growth promotant. The growth promotant may be a β-agonist, antibiotic, steroid or hormone. The composition is fed to the animal for a period of time before administration of the growth promotant, during administration of the growth promotant, and/or after administration of the growth promotant. Administration of the composition to the animal ameliorates, or prevents development of, at least one deleterious symptom or sign, such as a deleterious symptom or sign potentially associated with administration of the growth promotant. Embodiments of a composition comprising (i) a growth promotant and (ii) glucan, silica, mineral clay, mannans, or any combination thereof also are disclosed.

### CROSS-REFERENCE TO RELATED APPLICATIONS

This application claims the benefit of the earlier filing dates of U.S. Provisional Patent Application Nos. 61/932,146, 61/932,149 and 61/932,158, all filed on Jan. 27, 2014, and U.S. Provisional Patent Application No. 61/939,206, filed on Feb. 12, 2014, which are incorporated by reference herein in their entirety.

### FIELD

This invention concerns a composition and method for administration with a growth promotant.

### BACKGROUND

Animals intended for human consumption typically receive feed additives, supplements, and/or growth promotants to increase their weight and/or lean muscle mass. In some instances an animal exhibits signs or symptoms that may be attributed, anecdotally or otherwise, to a feed additive, supplement, or growth promotant administered to the animal. Concern arises when the animal's apparent welfare is negatively impacted, particularly if any of the actual or perceived deleterious impact reduces the quality and/or quantity of product obtained from the animal. Furthermore, products derived from animals receiving such additives, supplements and/or growth promotants may be deemed less valuable or even unsuitable for human consumption even though there may be no evidence of harmful effects from consumption of the products. While these additives, supplements and/or growth promotants are deemed vital to animal husbandry, so too are compositions and/or methods that ameliorate or prevent associated negative side effects.

### SUMMARY

This disclosure concerns embodiments of a composition (Composition I) comprising silica, mineral clay, mannans, or any combination thereof, and methods of administering the composition with a growth promotant to an animal. Composition I may further comprise glucan. This disclosure also concerns embodiments of a composition (Composition II) comprising (i) an embodiment of Composition I and (ii) a growth promotant, as well as methods of using and making Composition II. The growth promotant may be an antibiotic, steroid, hormone, β-agonist, or a combination thereof. In a particular embodiment, the growth promotant is a β-agonist. Hereinafter, unless otherwise specified or the context clearly indicates otherwise, the term “β-agonist” refers to a compound or combination of compounds, or salts or prodrugs thereof, that act on one or more of the β-adrenoreceptors.

In one embodiment, an animal is administered a growth promotant and an embodiment of Composition I. In another embodiment, the method includes identifying an animal to which a growth promotant will be administered or to which at least one dose of a growth promotant has been administered, and administering an embodiment of Composition I to the animal.

Composition I may be administered to the animal that has received or will receive a growth promotant for an effective period of time to (i) increase a feed intake of the animal relative to a feed intake of the animal prior to growth promotant administration; (ii) produce a greater weight gain of the animal relative to a weight gain of an animal that has not received the growth promotant; or (iii) both. Composition I may be administered to the animal for an effective period of time to (i) increase a feed intake of the animal relative to a feed intake of an animal that has received the growth promotant but has not received Composition I; (ii) increase a feed efficiency of the animal relative to an animal that has received the growth promotant but has not received Composition I; (iii) produce a greater weight gain of the animal relative to a weight gain of an animal that has received the growth promotant but has not received Composition I; (iv) produce a greater lean muscle gain of the animal relative to a lean muscle gain of an animal that has received the growth promotant but has not received Composition I; (v) produce an increased ratio of lean:fat gain of the animal relative to a ratio of lean:fat gain of an animal that has received the growth promotant but has not received Composition I; and/or (vi) produce a higher harvest value of the animal relative to a harvest value of an animal that has received the growth promotant but has not received Composition I; or (vii) any combination thereof.

Composition I may be administered to the animal before administration of a growth promotant, during administration of the growth promotant, and/or following administration of the growth promotant (e.g., during a withdrawal period). Composition I and/or the growth promotant may be administered to the animal daily. Composition I and the growth promotant may be administered simultaneously or sequentially in any order.

Administering Composition I and a growth promotant to the animal ameliorates at least one deleterious symptom or sign observed or measured in the animal and/or prevents development of a deleterious symptom or sign associated at least anecdotally with the growth promotant. Deleterious symptoms and signs include, but are not limited to, lameness, stiffness, muscle tremors, muscle damage, kidney damage, an increase or decrease in blood creatinine, an increase or decrease in blood glucose, an increase or decrease in relative and/or absolute weights of kidneys, heart and/or liver, an increase in signs of injury, a stress indicator, an aberrant immune system biomarker, an aberrant inflammation biomarker, or a combination thereof. Stress indicators may include, but are not limited to, increased panting, increased time lying down, increased temperature, increased sweating, increased heart rate, increased respiratory rate, respiratory alkalosis, decreased feed intake, increased water consumption, rumen acidosis, metabolic acidosis, dark cutters, poor carcass quality, decreased milk production, decreased immune function, or any combination thereof, compared to an animal that has not been administered the growth promotant. Immune system biomarkers include, but are not limited to, L-selectin, interleukin-1β (IL-1β), and antibody levels. Inflammation biomarkers include, but are not limited to, pro-inflammatory markers, such as COX-2, IL-1β, interleukin-6, tumor necrosis factor alpha (TNF-α), interleukin-8 receptor (IL8R), L-selectin, and macrophage inflammatory protein 1-alpha (MIP) gene expression.

Composition I may be admixed into a feedstuff that is subsequently fed to the animal. The growth promotant also may be admixed into a feedstuff that is subsequently fed to the animal. In some embodiments, Composition I and the growth promotant are admixed into a single feedstuff. Each feedstuff may be, for example, a feed ration, a mineral supplement, protein supplement, a premix, molasses, a liquid feed, or water.

In some embodiments, Composition I and a growth promotant are combined to provide a composition (Composition II). Composition II may have a ratio of Composition I to growth promotant that ranges from 0.01:1 to 20,000,000:1 by weight. Composition II may be formulated as a powder, a granule, a pellet, a solution, or a suspension. Composition II may include other components, for example, monensin, tylosin phosphate, melengestrol acetate, virginiamycin, or any combination thereof. Composition II may be admixed into a feedstuff in an amount sufficient to provide 0.1 kg to 100 kg per ton of feedstuff of Composition I and 0.0001 kg to 10 kg per ton of feedstuff of the growth promotant.

The foregoing and other objects, features, and advantages of the invention will become more apparent from the following detailed description, which proceeds with reference to the accompanying figures.

### BRIEF DESCRIPTION OF THE DRAWINGS

FIG. 1 provides Western blotting results demonstrating the effect of embodiments of a disclosed composition on the expression of neutrophil L-selectin as described in Example 1.

FIG. 2 provides Western blotting results demonstrating the effects of a disclosed embodiment of a composition in unheated and heated (pelleted) forms on the expression of neutrophil L-selectin as described in Example 2.

FIG. 3 is a graph summarizing the effects of a disclosed embodiment of a composition on the expression of the mRNA encoding L-selectin in rat neutrophils as described in Example 3.

FIG. 4 provides Western blotting results demonstrating the effects of a disclosed embodiment of a composition on the expression of neutrophil interleukin-1β (Il-1β) as described in Example 4.

FIG. 5 is a graph summarizing the effects of different compositions on the ability of rat neutrophils to affect the viability of Staphylococcus aureus bacteria as described in Example 8.

FIG. 6 is a graph summarizing the effects of different compositions on the expression of mRNA encoding interleukin-8 receptor in rat neutrophils as described in Example 8.

FIG. 7 is a graph summarizing the effects of different compositions on the expression of mRNA encoding L-selectin in rat neutrophils as described in Example 8.

FIG. 8 is a table providing the body weight (BW), average daily gain (ADG), dry matter intake (DMI and gain to feed ratio (G:F) of beef steers, illustrating the effects of administering Composition I in combination with ractopamine, as described in Example 14.

FIG. 9 is a table providing carcass characteristics of the beef steers from FIG. 8.

FIG. 10 is a table providing ultrasound data from the beef steers from FIG. 8.

### DETAILED DESCRIPTION

This disclosure concerns embodiments of a composition (Composition I) and a method for administering the composition and a β-agonist to an animal. Administration of Composition I with the β-agonist ameliorates or prevents at least one deleterious symptom or sign in the animal, such as a deleterious symptom or sign associated at least anecdotally with β-agonist administration.

### I. DEFINITIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Administering: Administration by any route to a subject. As used herein, administration typically but not necessarily refers to oral administration.

Co-administration: Administering two or more agents simultaneously or sequentially in any order to a subject to provide overlapping periods of time in which the subject is experiencing effects, beneficial and/or deleterious, from each agent. One or both of the agents may be a therapeutic agent. The agents may be combined into a single composition or dosage form, or they may be administered as separate agents.

Dark cutters: Cattle whose meat appears a dark red or purple when exposed to air compared to the more typical bright red or pinkish beef. Dark cutting results from stress, which depletes glycogen from the animal's muscle. After slaughter, glycolysis occurs in the muscle tissue. Glycogen is converted into lactic acid, which reduces the meat pH, e.g., from pH 7.2 in a live animal to a pH of 5.3 to 5.7. When the glycogen content in the muscle is low, the pH may remain above 5.8 and the meat appears dark. The meat also may lose more water during cooking, has a reduced shelf life, and has a sticky texture compared to red/pink beef.

Excipient: A physiologically inert substance that is used as an additive in a composition, such as a pharmaceutical composition. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Examples of excipients include but are not limited to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Feed efficiency: A measure of an animal's efficiency in converting feed mass into the desired output, e.g., weight gain, milk production. Feed efficiency also may be referred to as feed conversion ratio, feed conversion rate, or feed conversion efficiency.

Feedstuff: As used herein, the term “feedstuff” refers to anything that may be consumed by an animal. The term “feedstuff” encompasses solid and liquid animal feeds (e.g., a feed ration), supplements (e.g., a mineral supplement, a protein supplement), a premix, water, and feed additive carriers (e.g., molasses).

Glucocorticoid: A class of steroid hormones that bind to the glucocorticoid receptors in vertebrate animal cells. Exemplary endogenous glucocorticoids include cortisol (hydrocortisone) and corticosterone.

Mannans: A class of polysaccharides including the sugar mannose. The mannan family includes pure mannans (i.e., the polymer backbone consists of mannose monomers), glucomannans (the polymer backbone comprises mannose and glucose), and galactomannans (mannans or glucomannans in which single galactose residues are linked to the polymer backbone). Mannans are found in cell walls of some plant species and yeasts.

Mineral Clay: According to the AIPEA (Association Internationale pour l'Etude des Argiles (International Association for the Study of Clays)) and CMS (Clay Minerals Study) nomenclature committees, the term “mineral clay” refers to a mineral that imparts plasticity to a clay and hardens upon drying or firing. Mineral clays include aluminum silicates, such as aluminum phyllosilicates. Mineral clays usually include minor amounts of impurities, such as potassium, sodium, calcium, magnesium, and/or iron.

Pharmaceutically acceptable: The term “pharmaceutically acceptable” refers to a substance that can be taken into a subject without significant adverse toxicological effects on the subject.

Therapeutic agent: An agent that is capable of providing a therapeutic effect, e.g., preventing a disorder, inhibiting a disorder, such as by arresting the development of the disorder or its clinical symptoms, or relieving a disorder by causing regression of the disorder or its clinical symptoms.

Therapeutically effective amount: A quantity or concentration of a specified compound or composition sufficient to achieve a desired effect in a subject, e.g., a subject being treated for a disorder. The therapeutically effective amount may depend at least in part on the species of animal being treated, the size of the animal, and/or the severity of the disorder.

### II. COMPOSITION I

Embodiments of Composition I comprise silica, mineral clay, mannans, or any combination thereof. In some embodiments, Composition I further comprises glucan.

Composition I may further comprise an endoglucanohydrolase, such as β-1,3 (4)-endoglucanohydrolase. Suitable sources of silica include, but are not limited to, sand, quartz, diatomaceous earth, and synthetic silica. In certain embodiments, the mannans comprise glucomannan.

The components of Composition I are prepared by methods commonly known in the art and can be obtained from commercial sources. Components of Composition I (e.g., silica, mineral clay) also may be present in the environment. Diatomaceous earth is available as a commercially-available, acid-washed product comprising 95% silica (SiO₂) and with its remaining components not assayed but comprising primarily ash (minerals) as defined by the Association of Analytical Chemists (AOAC, 2002). The mineral clays (e.g., aluminosilicates) used in Composition I may be any of a variety of clays including, but not limited to, commercially available clays such as montmorillonite clay, bentonite and zeolite. Glucan and mannans can be obtained from plant cell walls, yeast (e.g., Saccharomyces cerevisiae, Candida utilis), certain fungi (e.g., mushrooms), and bacteria. In some embodiments, the glucans include soluble and/or insoluble β-glucans, such as (1,3/1,4) β-glucan (β-1,3 (4) glucan), (1,3/1,6) β-glucan, or a combination thereof. One commercial source of glucan and mannans (e.g., β-1,3 (4) glucan and glucomannan) is a yeast cell wall extract derived from inactivated yeast (Saccharomyces cerevisiae). The yeast cell wall extract may have a composition including 0-8% moisture, 92-100% dry matter, 10-55% protein, 0-25% fats, 0-2% phosphorus, 10-30% β-glucan, 0-25% mannans, and 0-5% ash. In one example, the yeast cell wall extract had the following composition: 2-3% moisture, 97-98% dry matter, 14-17% proteins, 20-22% fats, 1-2% phosphorus, 22-24% mannans, 24-26% β-1,3 (4) glucan, and 3-5% ash. β-1,3 (4)-endoglucanohydrolase may be produced from submerged fermentation of a strain of Trichoderma longibrachiatum.

Some embodiments of Composition I include 1-40 wt % silica, 1-25 wt % glucan and mannans, and 40-92 wt % mineral clay. In one embodiment, Composition I comprises 5-40 wt % silica, 2-15 wt % glucan and mannans, and 40-80 wt % mineral clay. In another embodiment, Composition I comprises 20-40 wt % silica, 4-10 wt % glucan and mannans, and 50-70 wt % mineral clay. In another embodiment, Composition I comprises 15-40 wt % silica, 1-15 wt % glucans, 0-10 wt % mannans, and 50-81 wt % mineral clay. In another embodiment, Composition I comprises 20-30 wt % silica, 1.0-3.5 wt % glucans, 1.0-6.0 wt % mannans, and 60-75 wt % mineral clay.

In some embodiments, β-glucans and mannans are obtained from yeast cell wall extract, and Composition I comprises 1-40 wt % silica, 1-30 wt % yeast cell wall extract, 40-92 wt % mineral clay. In one embodiment, Composition I comprises 10-40 wt % silica, 5-20 wt % yeast cell wall extract, and 40-80 wt % mineral clay. In another embodiment, Composition I comprises 15-30 wt % silica, 5-15 wt % yeast cell wall extract, and 55-70 wt % mineral clay.

In any of the above embodiments, Composition I may further comprise an endoglucanohydrolase, such as β-1,3 (4)-endoglucanohydrolase. Composition I may include from at least 0.05 wt % endoglucanohydrolase to 5 wt % endoglucanohydrolase, such as from 0.05-3 wt % β-1,3 (4)-endoglucanohydrolase. In one embodiment, Composition I consists essentially of 0.1-3 wt % β-1,3 (4)-endoglucanohydrolase, 20-40 wt % silica, 2-20 wt % glucan and mannans, and 50-70 wt % mineral clay.

In any of the above embodiments, the silica may be provided by diatomaceous earth. In any of the above embodiments, the glucans may be β-glucans. In any of the above embodiments, the mannans may comprise glucomannan. In one embodiment, Composition I consists essentially of 0.1-3 wt %, β-1,3 (4)-endoglucanohydrolase, 20-40 wt % diatomaceous earth, 2-20 wt % β-glucan and glucomannans, and 50-70 wt % mineral clay.

In one embodiment, Composition I comprises 0.1-1 wt % β-1,3 (4)-endoglucanohydrolase, 20-40 wt % diatomaceous earth, 5-20 wt % yeast cell wall extract, and 40-80 wt % mineral clay. In another embodiment, Composition I comprises 0.1-0.5 wt % β-1,3 (4)-endoglucanohydrolase, 20-30 wt % diatomaceous earth, 5-15 wt % yeast cell wall extract, and 60-70 wt % mineral clay. In still another embodiment, Composition I comprises 0.2 wt % β-1,3 (4)-endoglucanohydrolase, 24.5 wt % diatomaceous earth, 10.8 wt % yeast cell wall extract, and 63.9 wt % mineral clay.

In some embodiments, Composition I includes additional components. Additional components may be used for any desired purpose, such as a substantially biologically inert material added, for example, as a filler, or to provide a desired beneficial effect. For example, Composition I may include a carbonate (including a metal carbonate such as calcium carbonate), kelp, a vitamin (such as a niacin supplement or vitamin B-12 supplement), biotin, d-calcium pantothenate, choline chloride, thiamine mononitrate, pyridoxine hydrochloride, menadione dimethylpyrimidinol bisulfite, riboflavin-5-phosphate, folic acid, soybean oil, calcium aluminosilicate, rice hulls, mineral oil, or any combination thereof.

Composition I may be formulated in any suitable form, including a powder, a granule, a pellet, a solution, or a suspension. Certain disclosed embodiments are formulated as a dry, free-flowing powder. This powder is suitable for direct inclusion into a commercially-available feed, food product or as a supplement to a total mixed ration or diet. The powder may be mixed with either solid or liquid feed or with water. In another embodiment, Composition I is formed into pellets.

In some embodiments, Composition I has an average particle size selected to be compatible with a feedstuff or other components with which Composition I may be admixed, including a β-agonist. The term “compatible” as used herein means that the particle size is sufficiently similar to reduce or eliminate particle size segregation when Composition I is admixed with the feedstuff or other components. For example, if Composition I is admixed with a feedstuff or component having an average particle size of 50-200 μm, Composition I may have a similar average particle size, e.g., from 80-120% of the feedstuff/component particle size with which Composition I is admixed.

In one embodiment, when incorporated directly into feeds, Composition I may be added in amounts ranging from 0.1 to 100 kg per ton (2000 pounds) of feed. In some embodiments, Composition I is added in amounts ranging from 0.1 to 50 kg per ton or from 0.1 to 20 kg per ton of feed. In other embodiments, Composition I is added to animal feedstuffs or to food in amounts from 0.5 kg to 10 kg per ton of feed. In certain embodiments, Composition I may be added to feeds in amounts ranging from 1 to 5 kg per ton of feed.

When expressed as a percentage of dry matter of feed, Composition I may be added to animal feedstuffs or to foods in amounts ranging from 0.01 to 2.5% by weight, such as from 0.0125% to 2% by weight. In one embodiment, Composition I is added to animal feedstuffs or to food in amounts from 0.05 to 1.5% by weight, such as from 0.06% to 1% by weight. In another embodiment, Composition I is added in amounts from 0.1 to 0.7% by weight, such as from 0.125% to 0.5% by weight of feed.

Alternatively, Composition I may be fed directly to animals as a supplement in amounts of from greater than 0.01 gram to 20 gram per kilogram of live body weight, such as from 0.01 gram to 10 gram per kilogram of live body weight, from 0.01 gram to 1 gram per kilogram of live body weight, from 0.01 gram to 0.5 gram per kilogram of live body weight, or from 0.02 gram to 0.4 gram per kilogram of live body weight per day. In some embodiments, Composition I may be provided for use with many mammalian species in amounts of from 0.05 grams to 0.20 grams per kilogram of live body weight per day.

For cattle, Composition I may be provided in the range of from 10 grams per head per day to 70 grams per head per day, such as from 45 grams per head per day to 70 grams per head per day, or from 50 grams per head per day to 60 grams per head per day. A person of ordinary skill in the art will appreciate that the amount of Composition I fed can vary depending upon a number of factors, including the animal species, size of the animal and type of the feedstuff to which Composition I is added.

Typically, Composition I is administered daily to the animal at time intervals believed or determined to be effective for achieving a beneficial result. Composition I may be administered in a single dose daily or in divided doses throughout the day. In some instances, one or more individual components of Composition I may be administered to the animal at a first time, and remaining components may be administered individually or in combination at one or more subsequent times during the same day.

Without wishing to be bound by any particular theory of operation, Composition I may enhance the animal's immune system (i.e., the innate and/or adaptive immune system). For example, some embodiments of Composition I affect levels of immune biomarkers including, but not limited to, neutrophil L-selectin, IL-1β and/or gene expression of Crp, Mbl2, Apcs, Il5, Ifna1, Ccl12, Csf2, Il13, Il10, Gata3, Stat3, C3, Tlr3, Ccl5, Mx2, Nfkb1, Nfkbia, Tlr9, Cxcl10, Cd4, Il6, Ccl3, Ccr6, Cd40, Ddx58, Il18, Jun, Tnf, Traf6, Stat1, Ifnb1, Cd80, Tlr1, Tlr6, Mapk8, Nod2, Ccr8, Irak1, Cd1d1, Stat4, Ilr1, Faslg, Irf3, Ifnar1, Slc11a1, Tlr4, Cd86, Casp1, Ccr8, Icam1, Camp, Tlr7, Irf7, Rorc, Cd401g, Tbx21, Casp8, Il23a, Cd14, Cd8a, Cxcr3, Foxp3, Lbp, Mapk1, Myd88, Stat6, Agrin and/or IL33. As disclosed in U.S. Pat. No. 8,142,798, which is incorporated herein by reference, some embodiments of Composition I also augment an animal's adaptive immune system, e.g., by increasing response to a vaccine; antibody levels, such as IgG levels, may be increased, relative to an animal that has received a vaccine but has not been administered Composition I. Composition I also may reduce the effects of stress in the animal, potentially by ameliorating the effects of stress (e.g., heat stress, pregnancy stress, parturition stress, etc.) on the animal's immune system. Some embodiments of Composition I affect levels of inflammation biomarkers, e.g., COX-2, IL-1β, tumor necrosis factor alpha (TNF-α), interleukin-8 receptor (IL8R), and/or L-selectin.

### III. GROWTH PROMOTANTS

Growth promotants are used to help increase the efficiency of animal production, such as by increasing the rate of weight gain, improved feed efficiency and/or product output. A growth promotant may also increase the quality of a product, such as increase the quality of meat produced. Growth promotants can include, but are not limited to, β-agonists, antibiotics, antimicrobials, steroids and hormones. In some embodiments, a growth promotant may be a compound that has one or more other uses and is used as a growth promotant at a lower dose than the dose for the primary application. For example, an antibiotic or antimicrobial compound may also be useful as a growth promotant when used at a sub-therapeutic dose. Exemplary growth promotants include, but are not limited to, β-agonists such as ractopamine and zilpaterol, somatotropin such as bovine somatotropin (bST) and recombinant bovine somatotropin (rbST), ionophores such as monesin, lasalocid, laidlomycin, salinomycin and narasin, hormones such as oestrogen, progesterone, testosterone and analogs thereof, estradiol benzoate, oxytetracycline hydrochloride, arsanilic acid, 4-hydroxy-3-nitrobenzenearsonic acid, erythromycin thiocyanate, tylosin phosphate, melengestrol acetate, iodinated casein, ethopabate, oleandomycin, penicillin G procaine, chlortetracycline, sulfathiazole, bambermycins, bacitracin, virginiamycin, chlortetracycline calcium complex, or salt and/or combinations thereof.

### IV. BETA-AGONISTS

As used herein, the term “β-agonist” or “β-adrenergenic agonist” refers to a compound, or combination of compounds, or the salts or prodrugs thereof, known to those in the art or that are hereafter discovered, that act on one or more of the β-adrenoreceptors, including the β1, β2 and/or β3 receptors. Beta-agonist salts may include any pharmaceutically acceptable salt, including hydrogen halide salts, metal halide salts, phosphate salts, sulfonate salts, ammonium salts, etc. Acceptable salts may include salts of organic or inorganic acids, such as hydrochloride, hydrobromide, hydroiodide, carbonate, hydrogen carbonate, tartrate, mesylate, acetate, maleate, and oxalate salts.

A β-agonist prodrug is an active or inactive compound that is modified chemically through in vivo physiological action, such as hydrolysis, metabolism and the like, into an active β-agonist following administration of the prodrug to a subject. The term “prodrug” as used herein means the pharmacologically acceptable derivatives such as esters, amides and phosphates, such that the resulting in vivo biotransformation product of the derivative is the active β-agonist. Prodrugs preferably have excellent aqueous solubility, increased bioavailability and are readily metabolized into the active species in vivo. Prodrugs of β-agonists may be prepared by modifying functional groups present in the compound in such a way that the modifications are cleaved, either by routine manipulation or in vivo, to the parent compound. The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active β-agonist in vivo when the prodrug is administered to a subject. Since prodrugs often have enhanced properties relative to the active agent pharmaceutical, such as, solubility and bioavailability, the β-agonists disclosed herein can be delivered in prodrug form. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group.

In some embodiments, the β-agonist has one or more chiral centers, giving rise to stereoisomers. The β-agonist may be a mixture of stereoisomers, such that the mixture is a racemic, or non-racemic mixture. Alternatively, the β-agonist may be substantially one stereoisomer with only small amounts, if any, of other stereoisomers present, such as ≦5%, ≦3%, or ≦1% of other stereoisomers.

Examples of β-agonists include, but are not limited to, dobutamine, isoproterenol, xamoterol, epinephrine, salbutamol (albuterol), levosalbutamol (levalbuterol), fenoterol, formoterol, metaproterenol, salmeterol, terbutaline, clenbuterol, isoetarine, pirbuterol, procaterol, ritodrine, arbutamine, befunolol, bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol, zinterol, or combinations thereof. In some embodiments, the term β-agonist does not include ractopamine. In some embodiments, the term β-agonist does not include zilpaterol. In certain embodiments, the term β-agonist does not include either ractopamine or zilpaterol.

Administration of a β-agonist to an animal is typically performed to increase the animals' feed efficiency, thereby resulting in greater body weight gain, a greater growth rate, and/or a greater market value. Administration of the β-agonist, may also alter the type of gain experienced by an animal, e.g., increased lean mass and less fat mass.

A β-agonist is administered to animals for a period of time effective to achieve desired results, such as from 1 day to greater than 100 days prior to harvest or from 1-60 days prior to harvest. A β-agonist may also have a withdrawal period, such as from 1 day to 7 days, or it may have no withdrawal period.

A β-agonist may be administered by any effective method. Routes of administration include, but are not limited to, oral administration, intramuscular injection, intravenous injection, intradermal injection, subcutaneous injection, inhalation such as nasal or oral inhalation, rectal administration, transdermal administration, or combinations thereof. In some embodiments, a β-agonist is added to the animal's drinking water in a range from 0.1 mg to 1000 mg per liter of water, such as 0.5 mg to 500 mg per liter, or 1 mg to 100 mg per liter. In other embodiments, a β-agonist is added to the animal's feed, either directly or as part of a premix. In some embodiments, the β-agonist is admixed with feed such that the concentration of β-agonist is in a range of 0.0001 kg to 10 kg per ton of feed, such as 0.001 to 1 kg per ton, or 0.01 kg to 0.5 kg per ton. In other embodiments, the concentration of β-agonist in feed is in a range of 0.05 mg to 15,000 mg per kg of feed, such as 0.5 mg to 1,500 mg per kg, or 1 mg to 500 mg per kg. Alternatively, the amount of β-agonist to be administered is determined according to the live body weight of the animal, such as from 0.001 mg to a 1000 mg per kg live body weight, from 0.005 mg to 500 mg per kg, or from 0.01 mg to 100 mg per kg.

In general, a β-agonist composition is administered in a dosage form that provides an effective amount of the β-agonist. An “effective amount” is an amount sufficient to increase the rate of weight gain, improve feed efficiency, and/or increase carcass leanness in the animal.

For oral administration, a β-agonist may be admixed with suitable carriers or diluents commonly employed in animal husbandry. Typical carriers and diluents commonly employed in such feedstuffs include, by way of example and without limitation, corn meal, soybean meal, alfalfa meal, rice hulls, soybean mill run, cottonseed oil meal, bone meal, ground corn, corncob meal, sodium chloride, urea, cane molasses, and the like. Such carriers promote a uniform distribution of the active ingredient in the finished feed ration into which such compositions are added, thereby ensuring proper distribution of the active ingredient throughout the feed.

Although a β-agonist typically is administered via daily feed rations, it can be incorporated into salt blocks or mineral licks, and/or added directly to drinking water for convenient oral consumption. Additionally, a β-agonist can be formulated with polymorphous materials, waxes and the like for long-term controlled release, and administered to an animal as a bolus or tablet only as needed to maintain a desired daily dosage of active ingredient.

In some embodiments, a β-agonist is admixed with conventional carriers such as corn oil, sesame oil, carbowax, calcium stearate and the like. Such formulations can be molded into pellets and administered as an injection or as a slow-release subcutaneous implant. Such administrations can be made as often as needed to ensure proper dosing of active ingredient to obtain a desired rate of growth promotion and improvement in leanness and/or feed efficiency.

A β-agonist can be administered in combination with one or more other compounds known to have a beneficial effect upon animals. Typical compounds that might be co-administered with a β-agonist include antibiotics, for example any of the tetracyclines, tylosin, penicillins, cephalosporins, polyether antibiotics, glycopeptides, orthosomycins and related compounds commonly administered to swine, poultry, ruminants and the like.

In one embodiment, a β-agonist may be administered in combination with tylosin or a tetracycline. Such combinations will comprise the respective components in a ratio of 1-2 parts by weight of the β-agonist and 1-10 parts by weight of the partner component.

In one embodiment, a β-agonist is combined with an antibiotic, such as monensin (an antiprotozoal agent produced by Streptomyces cinnamonensis; (2R,3S,4R)-4-[(2R,5R,7S,8R,9S)-2-[(2R,5S)-5-ethyl-5-[(2S,3R,5S)-5-[(2S,3S,5R,6R)-6-hydroxy-6-(hydroxymethyl)-3,5-dimethyloxan-2-yl]-3-methyloxolan-2-yl]oxolan-2-yl]-7-hydroxy-2,8-dimethyl-1,10-dioxaspiro[4.5]decan-9-yl]-3-methoxy-2-methylpentanoic acid; Rumensin®, Elancoban®; Coban®):

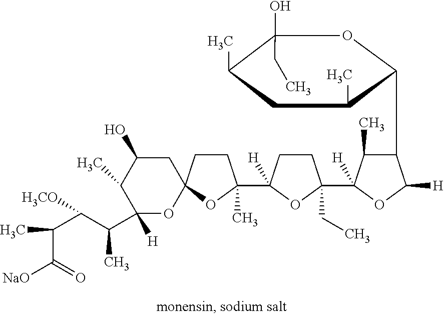

Monensin is admixed into feed at a final concentration of 10 grams to 40 grams per ton.

Other β-agonist combinations for use in animals may include:

(i) β-agonist, monesin (Rumensin®), and melengesterol acetate (17-hydroxy-6-methyl-16-methylenepregna-4,6-diene-3,20-dione acetate; Heifermax 500®, a steroidal progestin and antineoplastic agent);

(ii) β-agonist, monesin, melengesterol acetate, and tylosin phosphate (2-[(4R,5S,6S,7R,9R,11E,13E,15R,16R)-6-[(2R,3R,4R,5S,6R)-5-[(2S,4R,5S,6S)-4,5-dihydroxy-4,6-dimethyloxan-2-yl]oxy-4-(dimethylamino)-3-hydroxy-6-methyloxan-2-yl]oxy-16-ethyl-4-hydroxy-15-[[(2R,3R,4R,5R,6R)-5-hydroxy-3,4-dimethoxy-6-methyloxan-2-yl]oxymethyl]-5,9,13-trimethyl-2,10-dioxo-1-oxacyclohexadeca-11,13-dien-7-yl]acetaldehyde, phosphoric acid (Tylan®, Tylovet®), an antibiotic used against mycoplasma organisms); or

(iii) β-agonist, monesin, and tylosin phosphate.

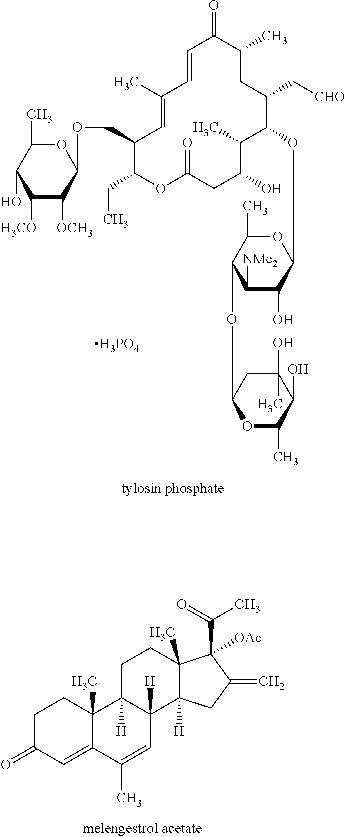

β-agonists also may be administered with virginiamycin, a streptogramin antibiotic used in animal feeds to prevent disease and improve growth.

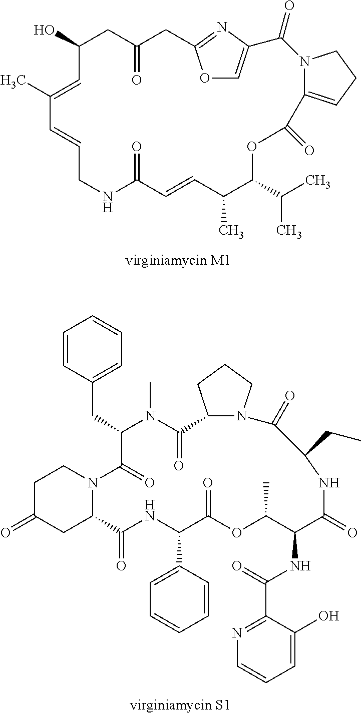 [SMILES: CC1=C\[C@@H](O)CC(=O)Cc2nc(co2)C(=O)N2CCC=C2C(=O)O[C@H](C(C)C)[C@H](C)/C=C/C(=O)NC\C=C\1 ; CC[C@H]1NC(=O)[C@@H](NC(=O)c2ncccc2O)[C@@H](C)OC(=O)[C@H](c2ccccc2)NC(=O)[C@@H]2CC(=O)CCN2C(=O)[C@H](Cc2ccccc2)N(C)C(=O)[C@@H]2CCCN2C1=O]

Factors affecting the dosage regimen may include, for example, the type (e.g., species and breed), age, size, sex, diet, activity, and condition of the animal; the type of administration used (e.g., oral via feed, oral via drinking water, subcutaneous implant, other parenteral route, etc.); pharmacological considerations, such as the activity, efficacy, pharmacokinetic, and toxicology profiles of the particular composition administered; and whether the β-agonist is being administered as part of a combination of active ingredients. Thus, the β-agonist amount can vary and, therefore, can deviate from the typical dosages. Determining such dosage adjustments is within the skill of a person of ordinary skill in the art using conventional means.

The β-agonist composition may be administered to the animal a single time. In general, however, the β-agonist composition is administered periodically over time. In some embodiments where the animal is a livestock animal, for example, a β-agonist is administered daily for at least 2 days, such as daily for 5 to 60 days. In certain embodiments, a β-agonist composition is administered daily for at least the last 2 days of a finishing period. The term “finishing period” refers to the later stage of the growing period for an animal. During this period, livestock animals are typically confined in a feedlot. In some embodiments where the livestock animal is a bovine animal, this period lasts for 90 to 225 days, and depends on, for example, the starting body weight of the animal. In some such embodiments, a β-agonist composition is administered daily for the last 5 days to the last 60 days of the finishing period. In other embodiments, a β-agonist is administered from the last 7 to the last 14 days, or for the last 14 days. Alternatively, a β-agonist composition is administered daily for the last 30 to 120 pounds of weight gain, or for the last 45 to 90 pounds of weight gain.

### V. RACTOPAMINE, RACTOPAMINE SALTS, AND RACTOPAMINE DERIVATIVES

Ractopamine, a β-adrenergic agonist (β-agonist), a ractopamine salt (e.g., ractopamine hydrochloride), and/or certain derivatives thereof have been administered to animals. Ostensibly, administration is performed to increase animals' feed efficiency, thereby resulting in greater body weight gain, a greater growth rate, and/or a greater market value. Administration of ractopamine, or a salt or derivative thereof, may also alter the type of gain experienced by an animal, e.g., increased lean mass and less fat mass. Synonyms for ractopamine include 1-(4-hydroxyphenyl)-2-[1-methyl-3-(4-hydroxyphenyl)propylamino]ethanol, 4-[3-[[2-hydroxy-2-(4-hydroxyphenyl)ethyl]amino]butyl]phenol, benzenemethanol 4-hydroxy-alpha-3-(4-hydroxyphenyl)-1-methylpropylaminomethyl, rac-ractopamine or (±)-ractopamine. Ractopamine exists in two diastereomeric forms resulting from the presence of two chiral carbons. The commercial preparation is a racemic mixture of the four stereoisomers RR, RS, SR, and SS with a minimal purity of about 96%. In some embodiments the four stereoisomers are present in about equal amounts, such as about 25%. In some other embodiments one or more stereoisomers are present in amounts greater than 25%. In certain embodiments, the mixture of stereoisomers may comprise less than all four of the stereoisomers, such as 1, 2 or 3 stereoisomers, including RR, SS, SR, RS, RR/SS, RR/SR, RR/RS, SS/SR, SS/RS, SR/RS, RR/SS/SR, RR/SS/RS, SS/SR/RS. A person of ordinary skill in the art will appreciate that there might still be trace amounts of the other isomers present, such as 5% or less, preferably 3% or less, more preferably 1% or less.

Ractopamine hydrochloride is marketed under the trade names Paylean® for swine, Topmax® for turkeys, and Optaflexx® for cattle, and is an FDA (Food and Drug Administration)-approved feed supplement. The RR isomer (butopamine) is a potent cardiostimulant in humans and has been shown to be the most active stereoisomer mediating the growth response in pigs.

| 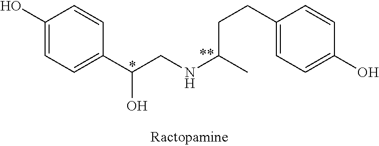 [SMILES: CC(CCc1ccc(O)cc1)NCC(O)c1ccc(O)cc1] | | |
|---|---|---|
| | Chiral sites | |
| Isomers | * | ** |
| RR | R | R |
| SR | S | R |
| RS | R | S |
| SS | S | S |

(*, ** indicate the chiral centers)

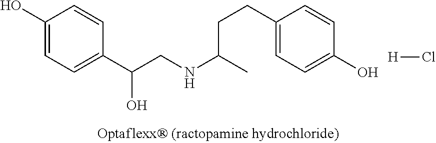

Ractopamine salts include any other pharmaceutically acceptable salt, including hydrogen halide salts, metal halide salts, phosphate salts, sulfonate salts, ammonium salts, etc. Acceptable salts may include salts of organic or inorganic acids, such as hydrochloride, hydrobromide, hydroiodide, carbonate, hydrogen carbonate, tartrate, mesylate, acetate, maleate, and oxalate salts.

Ractopamine derivatives may have a first general formula I:

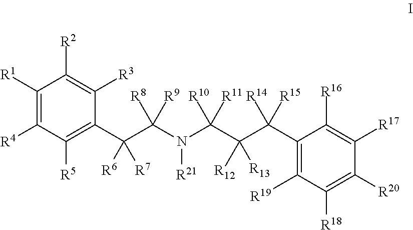

where R¹-R²¹ independently are hydrogen, hydroxyl, thiol, halogen, nitro, optionally substituted lower aliphatic, or optionally substituted lower heteroaliphatic. In some embodiments, R¹-R²⁰ are independently hydrogen, hydroxyl, halogen, nitro, optionally substituted lower aliphatic, or optionally substituted lower heteroaliphatic; and R²¹ is hydrogen or optionally substituted lower alkyl. In other embodiments, R¹-R⁵ and R¹⁶-R²⁰ are independently hydrogen, hydroxyl, halogen, nitro or optionally substituted lower alkyl; R⁶-R⁹ and R¹¹-R¹⁵ are independently hydrogen, hydroxyl, optionally substituted lower aliphatic, or optionally substituted lower heteroaliphatic; R¹⁰ is optionally substituted lower aliphatic, or optionally substituted lower heteroaliphatic; and R²¹ is hydrogen or optionally substituted lower alkyl. In certain embodiments, R¹-R⁹ and R¹¹-R²⁰ are independently hydrogen, hydroxyl or optionally substituted lower alkyl; R¹⁰ is optionally substituted lower alkyl; and R²¹ is hydrogen or optionally substituted lower alkyl. In certain other embodiments, at least one of R¹-R⁵, and at least one of R¹⁶-R²⁰, and at least one of R⁶-R¹⁵ are hydroxyls. The ractopamine derivative also may be a pharmaceutically acceptable salt.

In some embodiments, ractopamine derivatives have a general formula II:

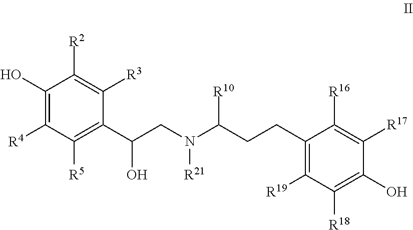

where R²-R⁵ and R¹⁶-R¹⁹ are independently hydrogen, hydroxyl, halogen or optionally substituted lower alkyl; R¹⁰ is lower alkyl; and R²¹ is hydrogen or lower alkyl. In certain embodiments R¹⁰ is methyl and R²¹ is hydrogen. Methods of making exemplary ractopamine derivatives according to general formula II are found, e.g., in U.S. Pat. No. 4,690,951 which is incorporated herein by reference.

Ractopamine is fed to animals for a period of time effective to achieve desired results. For example, this effective period of time typically is 1-60 days prior to harvest. Typically, ractopamine has a zero-day withdrawal period.

Ractopamine hydrochloride (typically administered as Optaflexx® or Optaflexx® 45) is typically administered to cattle as part of a complete feed or as a top dress, and is fed in an amount of from 70 to 430 mg per head per day for 28 to 42 days prior to harvest. For a complete feed, from 8 to 25 grams of ractopamine hydrochloride (90% dry matter basis) per ton of feedstuff, preferably from 8.2 to 24.6 grams per ton (i.e. from 9 ppm to 27 ppm), is fed continuously as a complete feed to provide from 70 to 400 mg per head per day for the last 28 to 42 days on feed. When provided as a top dress feed, ractopamine hydrochloride is admixed in a concentration of from 100 to 800 grams per ton of feedstuff, (i.e. from 70 to 400 mg per head per day of ractopamine hydrochloride (90% dry matter basis)) and a minimum of one pound of top dress Type C medicated feed per head per day is fed to the animals, for a maximum of 800 grams of ractopamine hydrochloride per ton of feedstuff, during the last 28-42 days on feed.

Ractopamine hydrochloride increases live weight by 22 pounds and hot carcass weight by 20 pounds when fed to cattle at 300 mg per head per day, compared to cattle that did not receive ractopamine hydrochloride. Ractopamine hydrochloride improves carcass leanness and yield grade, while having no effect on carcass quality, as measured by marbling score and quality grade. Yield grades estimate the amount of boneless, closely trimmed retail cuts from high-value parts of the carcass—the round, loin, rib, and chuck. However, they also reflect differences in the total yield of retail cuts. As defined by the United States Department of Agriculture, a Yield Grade 1 carcass yields the highest percentage of boneless, closely trimmed retail cuts, and/or higher cutability, while a Yield Grade 5 carcass has the lowest yield.

For swine, ractopamine hydrochloride (typically administered as Paylean® or Paylean® 9) is typically administered as part of a complete feed. From 0.25 to 2 pounds of Paylean® per ton of Type C medicated feed, preferably from 0.5 to 1 pound, is fed continuously as the sole ration to finishing swine weighing not less than 150 pounds for the last 45-90 pounds (group average) of weight gain prior to harvest, resulting in a ractopamine hydrochloride concentration in the feed from 3 grams per ton to 12 grams per ton, preferably from 4.5 grams to 9 grams per ton (from 5 ppm to 10 pm). Ractopamine hydrochloride increases average daily gain by 10%, result in 10% better feed efficiency, increase carcass weights by 5 pounds, and improve fat-free lean gain, while not affecting meat quality.

For turkeys, ractopamine hydrochloride (typically administered as TopMax® or Topmax® 9) is typically administered in an amount of from 0.25 to 1.5 pounds of Topmax® per ton of Type C medicated feed, preferably from 0.5 to 1.3 pounds, resulting in a ractopamine hydrochloride concentration in the feed from 4 grams per ton to 15 grams per ton, preferably from 4.5 grams per ton to 11.8 grams per ton (from 5 ppm to 13 ppm). The mix is fed continuously as the sole ration to finishing tom birds for the last 14 days, and to finishing hens for the last 7 to 14 days, prior to harvest. Ractopamine hydrochloride increases the average daily weight gain in toms by 13%, and by 19% in hens during the last 2 weeks. Ractopamine hydrochloride has known no effect on meat quality, including pH, color and tenderness.

Ractopamine may be administered by any effective method, but typically is administered orally. Other routes of administration can be employed, for instance intramuscular or intravenious injection. In some embodiments, ractopamine is added to the animal's drinking water. In other embodiments, ractopamine is added to the animal's feed, either directly or as part of a premix.

In general, ractopamine compositions are administered in a dosage form that provides an effective amount of ractopamine (e.g., ractopamine hydrochloride). An “effective amount” is an amount sufficient to increase the rate of weight gain, improve feed efficiency, and/or increase carcass leanness in the animal.

For oral administration, ractopamine is preferably admixed with suitable carriers or diluents commonly employed in animal husbandry. Typical carriers and diluents commonly employed in such feedstuffs include, by way of example and without limitation, corn meal, soybean meal, alfalfa meal, rice hulls, soybean mill run, cottonseed oil meal, bone meal, ground corn, corncob meal, sodium chloride, urea, cane molasses and the like. Such carriers promote a uniform distribution of the active ingredient in the finished feed ration into which such compositions are added, thereby ensuring proper distribution of the active ingredient throughout the feed.

While the preferred method for orally administering ractopamine is via the daily feed rations, it can be incorporated into salt blocks and mineral licks, as well as being added directly to drinking water for convenient oral consumption. Ractopamine can additionally be formulated with polymorphous materials, waxes and the like for long-term controlled release, and administered to an animal as a bolus or tablet only as needed to maintain the desired daily dosage of active ingredient.

For parenteral administration, ractopamine can be admixed with conventional carriers such as corn oil, sesame oil, carbowax, calcium stearate and the like. Such formulations can be molded into pellets and administered as an injection or as a slow-release subcutaneous implant. Such administrations can be made as often as needed to ensure the proper dosing of active ingredient to obtain the desired rate of growth promotion and improvement in leanness and feed efficiency.

Ractopamine can be administered in combination with other compounds known to have a beneficial effect upon animals. Typical compounds to be co-administered with ractopamine include antibiotics, for example any of the tetracyclines, tylosin, penicillins, cephalosporins, polyether antibiotics, glycopeptides, orthosomycins and related compounds commonly administered to swine, poultry, ruminants and the like. A preferred combination to be employed in the present method is an antibiotic such as tylosin or a tetracycline. Such combinations will comprise the respective components in a ratio of 1 to 2 parts by weight of ractopamine and 1 to 10 parts by weight of the partner component.

In one embodiment, ractopamine hydrochloride is combined with an antibiotic, such as monensin. For example, cattle may be fed as a sole ration a medicated feed comprising from 8 to 25 g/ton ractopamine hydrochloride and from 10 to 40 g/ton monensin, to provide from 70 to 430 mg per head per day ractopamine hydrochloride and from 0.1 to 0.5 mg, preferably from 0.14 to 0.42 mg, monensin per pound body weight, up to 480 mg per head per day monensin, for the last 28 to 42 days on feed.

Other approved ractopamine hydrochloride formulations for use in cattle include (i) ractopamine hydrochloride, monesin (Rumensin®), and melengesterol; (ii) ractopamine hydrochloride, monesin, melengesterol acetate, and tylosin phosphate; (iii) ractopamine hydrochloride, monesin, and tylosin phosphate. For swine, approved ractopamine hydrochloride formulations include ractopamine hydrochloride and tylosin phosphate. Ractopamine also may be administered with virginiamycin, a streptogramin antibiotic used in animal feeds to prevent disease and improve growth.

Factors affecting the dosage regimen may include, for example, the type (e.g., species and breed), age, size, sex, diet, activity, and condition of the animal; the type of administration used (e.g., oral via feed, oral via drinking water, subcutaneous implant, other parenteral route, etc.); pharmacological considerations, such as the activity, efficacy, pharmacokinetic, and toxicology profiles of the particular composition administered; and whether ractopamine is being administered as part of a combination of active ingredients. Thus, the ractopamine amount can vary, and, therefore, can deviate from the typical dosages set forth above. Determining such dosage adjustments is within the skill of a person of ordinary skill in the art using conventional means.

The ractopamine composition may be administered to the animal a single time. In general, however, the ractopamine composition is administered over time. In some embodiments where the animal is a livestock animal, for example, ractopamine is administered daily for at least 2 days, such as daily for 5 to 60 days, or daily for 28 to 42 days. In certain embodiments, the ractopamine composition is administered daily for at least the last 2 days of a finishing period. The term “finishing period” refers to the later stage of the growing period for an animal. During this period, livestock animals are typically confined in a feedlot. In some embodiments where the livestock animal is a bovine animal, this period lasts for 90 to 225 days, and depends on, for example, the starting body weight of the animal. In some such embodiments, the ractopamine composition is administered daily for the last 5 days to the last 60 days of the finishing period, or from the last 28 to the last 42 days of the finishing period. In other embodiments ractopamine hydrochloride is administered from the last 7 to the last 14 days, or for the last 14 days. Alternatively, the ractopamine composition is administered daily for the last 30 to 120 pounds of weight gain, or for the last 45 to 90 pounds of weight gain.

### VI. ZILPATEROL, ZILPATEROL SALTS, AND ZILPATEROL DERIVATIVES

Zilpaterol, a beta-adrenergic agonist (β-agonist), a zilpaterol salt (e.g., zilpaterol hydrochloride), and/or certain derivatives thereof have been administered to animals. A β-agonist is a compound that acts on one or more of the β-adrenoreceptors, including the β1,β2 and/or β3 receptors. Ostensibly, administration of a β-agonist is performed to increase animals' feed efficiency, thereby resulting in greater body weight gain, a greater growth rate, and/or a greater market value. Administration of zilpaterol, or a salt or derivative thereof, also may alter the type of gain experienced by an animal, e.g., increased lean mass and less fat mass. Synonyms for zilpaterol include 4,5,6,7-tetrahydro-7-hydroxy-6-(isopropylamino)imidazo[4,5,1-jk][1]benzazepin-2(1H)-one, (+/−)-trans-4,5,6,7-tetrahydro-7-hydroxy-6-(isopropylamino)imidazo[4,5,1-jk][1]benzazepin-2(1H)-one, 4,5,6,7-tetrahydro-7-hydroxy-6-[(1-methyl-ethyl)amino]-imidazo[4,5,1-jk][1]benzazepin-2(1H)-one, and RU-42173. Zilpaterol has two chiral carbons and consequently four optical enantiomers: (6R,7R), (6R,7S), (6S,7R), and (6S,7S). The hydrochloride salt of the (6S,7S) enantiomer is marketed under the trade name Zilmax (zilpaterol hydrochloride 4.8%, Merck Animal Health), and is an FDA (Food and Drug Administration)-approved feed supplement for cattle.

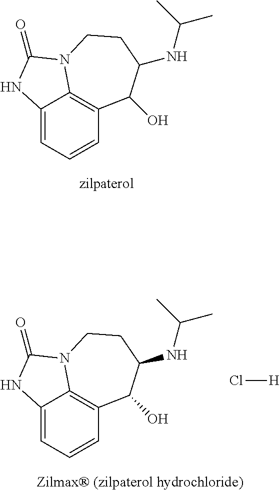 [SMILES: CC(C)NC1CCn2c(=O)[nH]c3cccc(c32)C1O ; CC(C)N[C@@H]1CCn2c(=O)[nH]c3cccc(c32)[C@H]1O ; Cl]

Zilpaterol salts include any other pharmaceutically acceptable salt, including hydrogen halide salts, metal halide salts, phosphate salts, sulfonate salts, ammonium salts, etc. Acceptable salts may include salts of organic or inorganic acids, such as hydrochloride, hydrobromide, carbonate, hydrogen carbonate, tartrate, mesylate, acetate, maleate, and oxalate salts.

Zilpaterol derivatives may have a first general formula I:

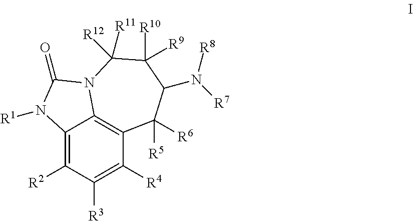

where R¹-R¹² independently are hydrogen, hydroxyl, thiol, halogen, optionally substituted lower aliphatic, or optionally substituted lower heteroaliphatic. In some embodiments, R¹-R⁴ and R⁸-R¹² are independently hydrogen or lower alkyl; R⁵ and R⁶ independently are hydrogen, hydroxyl or lower aliphatic; and R⁷ is hydrogen; C1-8 alkyl optionally substituted with hydroxy, C6-10 aryl; C6-10 heteroaryl; C6-10 aryloxy; C3-7 cycloalkyl; C3-7 cycloalkyl, optionally interrupted with a heteroatom (e.g., nitrogen) optionally substituted with C1-4 alkyl (e.g., methyl); or piperidinyl with the nitrogen optionally substituted by C1-4 alkyl. In certain embodiments, R¹-R⁴ and R⁸-R¹² are hydrogen, one of R⁵ and R⁶ is hydroxyl, and R⁷ is hydrogen or optionally substituted lower alkyl. The zilpaterol derivative also may be a pharmaceutically acceptable salt.

In some embodiments, zilpaterol derivatives have a general formula II:

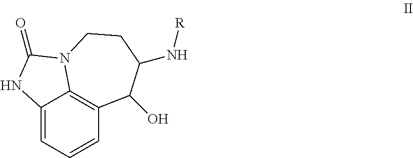

where R is hydrogen; C1-8 alkyl, optionally substituted with hydroxy; C6-10 aryl; C6-10 aryloxy; C3-7 cycloalkyl; C3-7 cycloalkyl optionally interrupted with a heteroatom (e.g., nitrogen), optionally substituted with C1-4 alkyl (e.g., methyl); or piperidinyl with the nitrogen optionally substituted by C1-4 alkyl. In some embodiments, R is methyl, ethyl, isopropyl, n-propyl, n-butyl, n-pentyl, 2,2-dimethylpropyl, 2-hydroxyethyl, hydroxymethyl, phenyl, phenoxy, cyclopropyl, cyclobutyl, cyclopentyl, cyclohexyl, or cycloheptyl. Methods of making exemplary zilpaterol derivatives according to general formula II are found, e.g., in U.S. Pat. No. 4,585,770 and U.S. Pat. No. 4,900,735, each of which is incorporated herein by reference.

Hereinafter, unless otherwise specified, the term “zilpaterol” refers to zilpaterol or a salt or derivative thereof. Zilpaterol is fed to animals for a period of time effective to achieve desired results. For example, this effective period of time typically is 1-60 days, such as 20-40 days, prior to harvest. Zilpaterol is withdrawn for a withdrawal period prior to harvest. The length of this withdrawal period may depend on, for example, the type (e.g., species and breed), age, weight, activity, and condition of the animal, as well as the maximum acceptable zilpaterol residue concentration in the meat of the animal. This withdrawal period typically is at least 3 days, such as for 3-10 days prior to harvest. In cattle, the withdrawal period is at least 3 days.

Zilpaterol hydrochloride is fed to cattle in an amount of 30-90 mg per head per day, typically 60-90 mg per head per day, for 20-40 days prior to harvest. Zilpaterol hydrochloride is indicated to increase carcass weight of cattle by 24 to 33 pounds, and increase live weight by 11 to 19 pounds, compared to cattle that did not receive zilpaterol hydrochloride. Zilpaterol hydrochloride is also indicated to increase the percentage of Yield Grade 1 cattle and cut the number of Yield Grade 4 and 5 cattle. In some instances zilpaterol hydrochloride administration may double the percentage of Yield Grade 1 cattle and cut in half the number of Yield Grade 4 and 5 cattle compared to cattle that have not received zilpaterol hydrochloride. Yield grades estimate the amount of boneless, closely trimmed retail cuts from high-value parts of the carcass—the round, loin, rib, and chuck. However, they also reflect differences in the total yield of retail cuts. As defined by the United States Department of Agriculture, a Yield Grade 1 carcass yields the highest percentage of boneless, closely trimmed retail cuts, and/or higher cutability, while a Yield Grade 5 carcass has the lowest yield. In one study, zilpaterol hydrochloride (administered as Zilmax®) was found to improve feed efficiency by 3%, and increase the value of cattle by $26.55/head, assuming a 31 lb. weight increase and a carcass price per pound of $1.55 (Intervet/Schering Plough Animal Health, 2010).

Zilpaterol may be administered by any effective method, but typically is administered orally. In some embodiments, zilpaterol is added to the animal's drinking water. In other embodiments, zilpaterol is added to the animal's feed, either directly or as part of a premix. In general, zilpaterol compositions are administered in a dosage form that provides an effective amount of zilpaterol (e.g., zilpaterol hydrochloride). An “effective amount” is an amount sufficient to increase the rate of weight gain, improve feed efficiency, and/or increase carcass leanness in the animal.

When the zilpaterol composition is orally administered, a daily dosage form is typically used. The total daily dose may be greater than 0.01 mg zilpaterol/kg body weight. In some embodiments, the daily dose is from 0.01 to 50 mg/kg, from 0.01 to 10 mg/kg, from 0.05 to 2 mg/kg, from 0.05 to 1 mg/kg, from 0.05 to 0.2 mg/kg, or from 0.05 to 0.2 mg/kg. In some embodiments where the zilpaterol is administered in the animal's feed, the concentration of zilpaterol in the feed (on a 90% dry matter basis) is at least 0.01 ppm (by weight). For bovine animals, the zilpaterol concentration may be no greater than about 75 ppm (by weight). In some embodiments, for example, the zilpaterol concentration is no greater than 38 ppm, from 0.5 to 20 ppm, from 3 to 8 ppm, or from 3.7 to 7.5 ppm (by weight). For swine animals, the zilpaterol concentration may be no greater than 45 ppm (by weight). In some such embodiments, for example, the concentration is no greater than 23 ppm, from 0.5 to 20 ppm, from 2 to 5 ppm, or from 2.2 to 4.5 ppm (by weight).

Although single oral daily doses are typically preferred, shorter or longer periods between doses can be used, depending on, for example, the animal's metabolism of zilpaterol. Smaller doses may be administered two or more times per day to achieve the desired total daily dose. Such multiple doses per day may, in some instances, be used to increase the total oral daily dose, if desired.

Suitable oral dosage forms include, for example, solid dosage forms (e.g., tablets, hard or soft capsules, granules, powders, etc.), pastes, and liquid dosage forms (e.g., solutions, suspensions, syrups, etc.). These dosage forms optionally comprise one or more suitable excipients, such as sweetening agents, flavoring agents, coloring agents, preservative agents, inert diluents (e.g., calcium carbonate, sodium carbonate, lactose, calcium phosphate, sodium phosphate, or kaolin), granulating and disintegrating agents (e.g., corn starch or alginic acid), binding agents (e.g., gelatin, acacia, or carboxymethyl cellulose), and lubricating agents (e.g., magnesium stearate, stearic acid, or talc). Liquid compositions will generally comprise a solvent, e.g., water or an aqueous solution, such as a buffer. The solvent preferably has sufficient chemical properties and quantity to keep the zilpaterol composition solubilized at normal storage temperatures, e.g., ambient temperatures. In some instances, the zilpaterol composition may comprise one or more preservatives.

In some embodiments, the zilpaterol is in the form of particles adhered to a support, which is fed to the animal. The supported zilpaterol may be incorporated into the animal's feed, either directly or as part of a premix. Contemplated supports include, for example, inert supports, such as calcium carbonate, limestone, oyster shell flour, talc, soybean hulls, soybean meal, soybean feed, soybean mill run, wheat middlings, rice hulls, corn meal, corn germ meal, corn gluten, starch, sucrose, and lactose. Other suitable supports include corn cob supports. The zilpaterol particles that are adhered to the support have a particle size that is less than the size of the support. Thus, for example, in some embodiments in which the support is from 300 to 800 μm, the zilpaterol particles (or at least about 95% of the zilpaterol particles) are less than 250 μm, such as from 50 μm to 200 μm. In certain embodiments, zilpaterol hydrochloride is provided as a premix comprising zilpaterol hydrochloride particles are affixed to ground corn cobs at a final concentration of 4.8% by weight.

To the extent zilpaterol is incorporated into feed, the feed mixture will vary depending on, for example, the type (e.g., species and breed), age, weight, activity, and condition of the intended recipient. For bovine and swine, various feeds are well known in the art, and often comprise cereals; sugars; grains; arachidic, tournsole, and soybean press cake; flours of animal origin, such as fish flour; amino acids; mineral salts; vitamins; antioxidants; etc. In general, the zilpaterol composition can be incorporated into any feed that is available and used for the animal. When incorporated into feed for cattle, zilpaterol hydrochloride is typically incorporated in an amount from 68 g/ton (2,000 pounds) of feed to 680 g/ton of feed (90% dry matter), or 75-750 g/metric ton, where sufficient feed is administered to the cattle to provide 60-90 mg zilpaterol hydrochloride per head per day. Zilpaterol hydrochloride is commercially available in pelleted form, and the pellets can be mixed with feed to provide the desired concentration of zilpaterol. Zilpaterol hydrochloride also can be added to liquids, typically within a pH from 3.5 to 7.5, such as liquid feed supplements (pH 3.8-7.5). Zilpaterol hydrochloride is added to such liquids at a typical concentration of 83-830 g/metric ton, where sufficient liquid is administered to provide 60-90 mg zilpaterol hydrochloride per head of cattle per day.

In one embodiment, zilpaterol hydrochloride is combined in feed at a final concentration of 6.8 g/ton with an antibiotic, such as monensin. For example, a medicated feed comprising 68-680 g/ton zilpaterol hydrochloride and 100-4000 g/ton monensin may be mixed with 1800-1980 pounds of unmedicated feed to form a feed with suitable levels of zilpaterol hydrochloride and monensin. In another embodiment, melengestrol acetate, a hormone that suppresses estrus, may be included at a concentration of 0.125-1.0 mg/lb of feed. Zilpaterol hydrochloride also may be provided as a supplement that can be mixed with feed, e.g., Zilmax® Supplement Z480, which includes 0.24 g zilpaterol per pound, ≧12.0% crude protein, ≧1.0% crude fat, ≧14.0% crude fiber, and 5.5-6.6% calcium (available from Hubbard Feeds, Mankato, Minn.). Approved zilpaterol hydrochloride formulations for use in cattle include (i) zilpaterol hydrochloride, monensin (Rumensie), and tylosin phosphate; (ii) zilpaterol hydrochloride and monensin; (iii) zilpaterol hydrochloride and melengestrol acetate; (iv) zilpaterol hydrochloride, monensin, and melengestrol acetate, and (v) zilpaterol hydrochloride, monensin, melengestrol acetate, and tylosin phosphate. Zilpaterol also may be administered with virginiamycin, a streptogramin antibiotic used in animal feeds to prevent disease and improve growth.

Zilpaterol compositions also may be administered via non-oral routes, such as rectally, via inhalation (e.g., via a mist or aerosol), transdermally (e.g., via a transdermal patch), or parenterally (e.g., subcutaneous injection, intravenous injection, intramuscular injection, implanted device, partially implanted device, etc.). In some embodiments, a zilpaterol composition is administered via an implant, such as a subcutaneous implant.

When administered via a subcutaneous implant, the total daily dose of zilpaterol is typically greater than 0.05 mg/kg body weight, particularly for bovine and swine animals. In some such embodiments, the daily dose is from 0.1 to 0.25 mg/kg.

If the zilpaterol composition is administered parenterally via an injection, the concentration of zilpaterol in the dosage form desirably is sufficient to provide the desired therapeutically effective amount of zilpaterol in a volume that is acceptable for parenteral administration. As with oral feeding, an injection dosage form may be administered once per day, although it is contemplated that shorter or longer periods between doses also could be used.

Factors affecting the dosage regimen may include, for example, the type (e.g., species and breed), age, size, sex, diet, activity, and condition of the animal; the type of administration used (e.g., oral via feed, oral via drinking water, subcutaneous implant, other parenteral route, etc.); pharmacological considerations, such as the activity, efficacy, pharmacokinetic, and toxicology profiles of the particular composition administered; and whether zilpaterol is being administered as part of a combination of active ingredients. Thus, the zilpaterol amount can vary, and, therefore, can deviate from the typical dosages set forth above. Determining such dosage adjustments is generally within the skill of those in the art using conventional means. The zilpaterol composition may be administered to the animal a single time. In general, however, the zilpaterol composition is administered over time. In some embodiments where the animal is a livestock animal, for example, zilpaterol is administered daily for at least 2 days, such as daily for 10 to 60 days or daily for 20 to 40 days. In certain embodiments, the zilpaterol composition is administered daily for at least the last 2 days of a finishing period. The term “finishing period” refers to the later stage of the growing period for an animal. During this period, livestock animals are typically confined in a feedlot. In some embodiments where the livestock animal is a bovine animal, this period lasts for 90 to 225 days, and depends on, for example, the starting body weight of the animal. In some such embodiments, the zilpaterol composition is administered daily for the last 10 days to the last 60 days of the finishing period, or from the last 20 to the last 40 days of the finishing period. There is typically a withdrawal period following the finishing period in which no zilpaterol is administered.

### VII. COMPOSITIONS COMPRISING COMPOSITION I AND A β-AGONIST

In some embodiments, Composition II comprises (i) an embodiment of Composition I and (ii) a β-agonist. Thus, Composition II comprises (i) silica, mineral clay, mannans, or any combination thereof, and (ii) a β-agonist. In some embodiments, Composition II further comprises glucan. In other embodiments, Composition II may further comprise an endoglucanohydrolase, such as β-1,3 (4)-endoglucanohydrolase. In some embodiments, Composition II comprises glucan, silica, mineral clay, mannans, and a β-agonist. In some embodiments, Composition II comprises 1-40 wt % silica, 1-25 wt % glucan and mannans, and 40-92 wt % mineral clay. Composition II may further include a therapeutic or otherwise active ingredient such as an antibiotic (such as monensin, virginiamycin, or tylosin phosphate), melengestrol acetate, or any combination thereof. Composition II may further include additional components for any desired purpose, such as a biologically inert filler. Exemplary additional components include, but are not limited to, a carbonate (including a metal carbonate such as calcium carbonate), kelp, a vitamin (such as a niacin supplement or vitamin B-12 supplement), biotin, d-calcium pantothenate, choline chloride, thiamine mononitrate, pyridoxine hydrochloride, menadione dimethylpyrimidinol bisulfite, riboflavin-5-phosphate, folic acid, soybean oil, calcium aluminosilicate, rice hulls, mineral oil, or any combination thereof.

In certain embodiments, the β-agonist is provided as a premix comprising the β-agonist and ground corn cobs at a final concentration such that when the premix is admixed with feed, the animal is administered a therapeutically effective amount of the β-agonist.

Composition II may be formulated in any suitable form, including a powder, a granule, a pellet, a solution, or a suspension. In some embodiments, Composition II is formulated as a dry, free-flowing powder. This powder is suitable for direct inclusion into a commercially-available feed, food product or as a supplement to a total mixed ration or diet. The powder may be mixed with either solid or liquid feed or with water. In another embodiment, Composition II is formed into pellets.

Composition II also may be formulated as a pharmaceutical composition. For oral administration, Composition II may be formulated in solid dosage forms (e.g., tablets, hard or soft capsules, granules, powders, etc.), pastes, and liquid dosage forms (e.g., solutions, suspensions, syrups, etc.). These dosage forms optionally comprise one or more suitable excipients, such as sweetening agents, flavoring agents, coloring agents, preservative agents, inert diluents (e.g., calcium carbonate, sodium carbonate, lactose, calcium phosphate, sodium phosphate, or kaolin), granulating and disintegrating agents (e.g., corn starch or alginic acid), binding agents (e.g., gelatin, acacia, or carboxymethyl cellulose), and lubricating agents (e.g., magnesium stearate, stearic acid, or talc). Liquid compositions will generally comprise a solvent, e.g., water or an aqueous solution, such as a buffer. The solvent preferably has sufficient chemical properties and is used in a sufficient quantity to keep the β-agonist and Composition I solubilized and/or suspended at normal storage temperatures, e.g., ambient temperatures. In some instances, the pharmaceutical composition may comprise one or more preservatives.

In some embodiments, Composition II, or a pharmaceutical composition comprising Composition II, may be admixed with feed to provide 0.1-20 kg per ton of feed of an embodiment of Composition I, and 0.0001-10 kg per ton of feed of β-agonist. In other embodiments, Composition II, or a pharmaceutical composition comprising Composition II, may be formulated to provide 0.01-20 g/kg live body weight of an embodiment of Composition I and 0.001-1000 mg/kg live body weight of β-agonist. Thus, Composition II may include Composition I and β-agonist in a ratio from 0.01:1 to 20,000,000:1 by weight. In some embodiments, Composition II includes Composition I and β-agonist in a ratio from 0.1:1 to 2,000,000:1, such as from 1:1 to 1,000,000:1, 10:1 to 500,000:1, 100:1 to 50,000:1, 200:1 to 10,000:1, 300:1 to 5,000:1, 400:1 to 2000:1, or 450:1 to 1200:1.

### VIII. COMPOSITIONS COMPRISING COMPOSITION I AND RACTOPAMINE

In some embodiments, Composition II comprises (i) an embodiment of Composition I and (ii) ractopamine. Thus, Composition II comprises (i) silica, mineral clay, mannans, or any combination thereof, and (ii) ractopamine. In some embodiments, Composition I further comprises glucan. In other embodiments, Composition II may further comprise an endoglucanohydrolase, such as β-1,3 (4)-endoglucanohydrolase. In some embodiments, Composition II comprises glucan, silica, mineral clay, mannans, and ractopamine. In some embodiments, Composition II comprises 1-40 wt % silica, 1-25 wt % glucan and mannans, and 40-92 wt % mineral clay. Composition II may further include a therapeutic or otherwise active ingredient such as an antibiotic (such as monensin, virginiamycin, or tylosin phosphate), melengestrol acetate, or any combination thereof. Composition II may further include additional components for any desired purpose, such as a biologically inert filler. Exemplary additional components include, but are not limited to, a carbonate (including a metal carbonate such as calcium carbonate), kelp, a vitamin (such as a niacin supplement or vitamin B-12 supplement), biotin, d-calcium pantothenate, choline chloride, thiamine mononitrate, pyridoxine hydrochloride, menadione dimethylpyrimidinol bisulfite, riboflavin-5-phosphate, folic acid, soybean oil, calcium aluminosilicate, rice hulls, mineral oil, or any combination thereof.

In certain embodiments, the ractopamine is provided as ractopamine hydrochloride. In one embodiment, the ractopamine is provided as a premix comprising ractopamine hydrochloride particles affixed to ground corn cobs at a final concentration of 45.4 grams per pound, or 100 grams per kilogram. In another embodiment the ractopamine is provided as a premix comprising ractopamine hydrochloride particles affixed to ground corn cobs at a final concentration of 9 grams per pound, or 20 grams per kilogram.

Composition II may be formulated in any suitable form, including a powder, a granule, a pellet, a solution, or a suspension. In some embodiments, Composition II is formulated as a dry, free-flowing powder. This powder is suitable for direct inclusion into a commercially-available feed, food product or as a supplement to a total mixed ration or diet. The powder may be mixed with either solid or liquid feed or with water. In another embodiment, Composition II is formed into pellets.

Composition II also may be formulated as a pharmaceutical composition. For oral administration, Composition II may be formulated in solid dosage forms (e.g., tablets, hard or soft capsules, granules, powders, etc.), pastes, and liquid dosage forms (e.g., solutions, suspensions, syrups, etc.). These dosage forms optionally comprise one or more suitable excipients, such as sweetening agents, flavoring agents, coloring agents, preservative agents, inert diluents (e.g., calcium carbonate, sodium carbonate, lactose, calcium phosphate, sodium phosphate, or kaolin), granulating and disintegrating agents (e.g., corn starch or alginic acid), binding agents (e.g., gelatin, acacia, or carboxymethyl cellulose), and lubricating agents (e.g., magnesium stearate, stearic acid, or talc). Liquid compositions will generally comprise a solvent, e.g., water or an aqueous solution, such as a buffer. The solvent preferably has sufficient chemical properties and is used in a sufficient quantity to keep the ractopamine and Composition I solubilized and/or suspended at normal storage temperatures, e.g., ambient temperatures. In some instances, the pharmaceutical composition may comprise one or more preservatives.

Composition II, or a pharmaceutical composition comprising Composition II, may be admixed with feed to provide 0.1-20 kg per ton of feed of an embodiment of Composition I, and 0.001-1 kg per ton of feed of ractopamine. Thus, Composition II may include Composition I and ractopamine in a ratio from 0.1:1 to 20,000:1 per ton of feed. In some embodiments, Composition II includes Composition I and ractopamine in a ratio from 0.5:1 to 10,000:1, such as from 1:1 to 5,000:1, 10:1 to 1,000:1, 50:1 to 500:1, or 100:1 to 200:1. In another embodiment, Composition II is admixed with a feedstuff in an amount sufficient to provide 0.01 to 2.5% by weight Composition I and at least 0.01 ppm by weight ractopamine, such as from 0.0125 to 2.5% by weight Composition I and 0.5 to 75 ppm by weight ractopamine, or from 0.125 to 0.5% by weight Composition I and 5 to 30 ppm by weight ractopamine. For cattle, Composition II, or a pharmaceutical composition comprising Composition II, may be formulated to provide 10-70 g of Composition I and 70-430 mg of ractopamine hydrochloride per head per day. For swine and poultry, Composition II, or a pharmaceutical composition comprising Composition II, may be formulated to provide 5 pounds per ton of feed of Composition I and from 4 to 12 grams per ton of feed of ractopamine.

### IX. COMPOSITIONS COMPRISING COMPOSITION I AND ZILPATEROL

In some embodiments, Composition II comprises (i) an embodiment of Composition I and (ii) zilpaterol, a zilpaterol salt, or a zilpaterol derivative (hereinafter “zilpaterol”). Thus, Composition II comprises (i) silica, mineral clay, mannans, or any combination thereof, and (ii) zilpaterol. Composition II may further comprise glucan, such as β-glucan, and/or an endoglucanohydrolase, such as β-1,3 (4)-endoglucanohydrolase. In some embodiments, Composition II comprises glucan, silica, mineral clay, mannans, and zilpaterol. In some embodiments, Composition II comprises 1-40 wt % silica, 1-25 wt % glucan and mannans, and 40-92 wt % mineral clay. Composition II may further include an antibiotic (such as monensin, tylosin phosphate, or virginiamycin), melengestrol acetate, or any combination thereof. Composition II may further include additional components for any desired purpose, such as a biologically inert filler. Exemplary additional components include, but are not limited to, a carbonate (including a metal carbonate such as calcium carbonate), kelp, a vitamin (such as a niacin supplement or vitamin B-12 supplement), biotin, d-calcium pantothenate, choline chloride, thiamine mononitrate, pyridoxine hydrochloride, menadione dimethylpyrimidinol bisulfite, riboflavin-5-phosphate, folic acid, soybean oil, calcium aluminosilicate, rice hulls, mineral oil, or any combination thereof.

In certain embodiments, the zilpaterol is provided as zilpaterol hydrochloride. In one embodiment, the zilpaterol is provided as a premix comprising zilpaterol hydrochloride particles affixed to ground corn cobs at a final concentration of 4.8% by weight. In another embodiment, zilpaterol is provided as a supplement comprising 0.24 g zilpaterol per pound, ≧12.0% crude protein, ≧1.0% crude fat, ≧14.0% crude fiber, and 5.5-6.6% calcium.

Composition II may be formulated in any suitable form, including a powder, a granule, a pellet, a solution, or a suspension. In some embodiments, Composition II is formulated as a dry, free-flowing powder. This powder is suitable for direct inclusion into a commercially-available feed, food product or as a supplement to a total mixed ration or diet. The powder may be mixed with either solid or liquid feed or with water. In another embodiment, Composition II is formed into pellets.

Composition II also may be formulated as a pharmaceutical composition. For oral administration, Composition II may be formulated in solid dosage forms (e.g., tablets, hard or soft capsules, granules, powders, etc.), pastes, and liquid dosage forms (e.g., solutions, suspensions, syrups, etc.). These dosage forms optionally comprise one or more suitable excipients, such as sweetening agents, flavoring agents, coloring agents, preservative agents, inert diluents (e.g., calcium carbonate, sodium carbonate, lactose, calcium phosphate, sodium phosphate, or kaolin), granulating and disintegrating agents (e.g., corn starch or alginic acid), binding agents (e.g., gelatin, acacia, or carboxymethyl cellulose), and lubricating agents (e.g., magnesium stearate, stearic acid, or talc). Liquid compositions will generally comprise a solvent, e.g., water or an aqueous solution, such as a buffer. The solvent preferably has sufficient chemical properties and quantity to keep the zilpaterol and Composition I solubilized and/or suspended at normal storage temperatures, e.g., ambient temperatures. In some instances, the pharmaceutical composition may comprise one or more preservatives.

Composition II, or a pharmaceutical composition comprising Composition II, may be formulated to provide 0.01-20 g/kg live body weight of an embodiment of Composition I and 0.01-50 mg/kg live body weight of zilpaterol. Thus, Composition II may include Composition I and zilpaterol in a ratio from 0.2:1 to 2,000,000:1 by weight. In some embodiments, Composition II includes Composition I and zilpaterol in a ratio from 1:1 to 1,000,000:1, such as from 10:1 to 500,000:1, 100:1 to 50,000:1, 200:1 to 10,000:1, 300:1 to 5,000:1, 400:1 to 2000:1, or 450:1 to 1200:1. In one embodiment, Composition II, or a pharmaceutical composition comprising Composition II, is admixed with a feedstuff in an amount sufficient to provide 0.01 to 2.5% by weight Composition I and 0.0075 to 0.075% by weight zilpaterol, such as from 0.125 to 0.5% by weight composition I and 0.0075 to 0.075% by weight zilpaterol. In another embodiment, Composition II is admixed with a feedstuff in an amount sufficient to provide 0.01 to 2.5% by weight Composition I and at least 0.01 ppm by weight zilpaterol, such as from 0.0125 to 2.5% by weight Composition I and 0.5 to 75 ppm by weight zilpaterol, or from 0.125 to 0.5% by weight Composition I and 0.5 to 20 ppm by weight zilpaterol. For cattle, Composition II, or a pharmaceutical composition comprising Composition II, may be formulated to provide 10-70 g of Composition I and 60-90 mg of zilpaterol hydrochloride per head per day.

### X. METHODS AND BENEFITS OF CO-ADMINISTRATION

An embodiment of Composition I is administered to an animal to which a growth promotant will be administered, or to which at least one dose of a growth promotant has been administered. The growth promotant may be a β-agonist, and in some embodiments, the β-agonist is ractopamine. In other embodiments, the β-agonist is zilpaterol. In some embodiments the animal is a food animal. In certain embodiments, the animal is a land animal, an aquatic animal or an amphibian. For example, the animal may be a mammal, an avian species, a fish, a reptile or a crustacean. In some embodiments, the animal is a ruminant animal (e.g., bovine, sheep, goat, deer, bison, buffalo, elk), a non-ruminant animal (e.g., swine, horse), or a poultry species (e.g., chicken, quail, turkey, duck, goose). In certain embodiments, the animal is an animal intended for consumption (i.e., a livestock animal), such as a bovine, swine, chicken or turkey.

Co-administration of Composition I and a growth promotant benefits the animal's health and/or welfare. Composition I, when administered to the animal, ameliorates or helps improve at least one deleterious symptom or sign observed or measured in the animal. In some instances, a deleterious symptom or sign may be attributed, anecdotally or otherwise, to a growth promotant. In some embodiments, administering Composition I to the animal prevents development of a deleterious symptom or sign associated with the growth promotant administration. Deleterious symptoms and signs may include, but are not limited to, lameness, stiffness, muscle tremors, muscle damage, kidney damage, an increase or decrease in blood creatinine, an increase or decrease in blood glucose, an increase or decrease in relative and/or absolute weights of kidneys, heart and/or liver, an increase in signs of injury, a stress indicator, an aberrant immune system biomarker, an aberrant inflammation biomarker, or a combination thereof. Stress indicators include, but are not limited to, increased panting, increased lying down, increased temperature, increased sweating, increased heart rate, increased respiratory rate, respiratory alkalosis, decreased feed intake, increased water consumption, rumen acidosis, metabolic acidosis, dark cutters, poor carcass quality, decreased milk production, decreased immune function, decreased overall health, and/or elevated stress hormone levels (e.g., glucocorticoids such as cortisol (hydrocortisone) and/or corticosterone). Immune system biomarkers include, but are not limited to, L-selectin, IL-1β, antibodies (e.g., IgG antibodies) and gene expression of Crp, Mbl2, Apcs, Il5, Ifna1, Ccl12, Csf2, Il13, Il10, Gata3, Stat3, C3, Tlr3, Ccl5, Mx2, Nfkb1, Nfkbia, Tlr9, Cxcl10, Cd4, Il6, Ccl3, Ccr6, Cd40, Ddx58, Il18, Jun, Tnf, Traf6, Stat1, Ifnb1, Cd80, Tlr1, Tlr6, Mapk8, Nod2, Ccr8, Irak1, Cd1d1, Stat4, Ilr1, Faslg, Irf3, Ifnar1, Slc11a1, Tlr4, Cd86, Casp1, Ccr8, Icam1, Camp, Tlr7, Irf7, Rorc, Cd401g, Tbx21, Casp8, Il23a, Cd14, Cd8a, Cxcr3, Foxp3, Lbp, Mapk1, Myd88, Stat6, Agrin and/or IL33. Inflammation biomarkers include, but are not limited to, COX-2, IL-1β, TNF-α, IL8R, and/or L-selectin.

In some embodiments, co-administration of Composition I and a growth promotant reduces a stress hormone level in the animal by at least 5%, at least 10%, at least 15%, at least 20%, or at least 25%, such as from 5-50%, from 5-25%, from 5-20%, or from 5-10%, compared to an average stress hormone level in biomarker in an animal that has received the growth promotant but has not received Composition I.

Co-administration of Composition I and a growth promotant may produce a concomitant change in a level of an immune system biomarker or an inflammation biomarker in an animal by at least 5%, at least 10%, at least 20%, at least 30%, at least 50%, at least 75%, at least 100%, at least 200%, or at least 500%, such as from 5-600%, from 10-500%, from 10-200%, or from 10-100%, compared to an average level of the biomarker in an animal that has received the growth promotant but has not received Composition I. The change may be an increase or a decrease, depending on the particular biomarker.

In some embodiments, a biomarker level is assessed by measuring a concentration of messenger RNA (mRNA) encoding the biomarker. Co-administration of Composition I and a growth promotant may produce a concomitant change in a concentration of mRNA encoding an immune system biomarker or an inflammation biomarker in an animal by at least 5%, at least 10%, at least 20%, at least 30%, at least 50%, at least 75%, at least 100%, or at least 200%, or at least 500%, such as from 5-600%, from 10-500%, from 10-200%, or from 10-100% compared to an average concentration of the mRNA in an animal that has received the growth promotant but has not received Composition I. The change may be an increase or a decrease, depending on the particular mRNA.

Co-administration of Composition I and a growth promotant also may benefit the animal's growth, weight gain, feed efficiency, value, and/or meat quality. In some embodiments, co-administering Composition I and the growth promotant to the animal produces a growth rate, weight gain, lean muscle gain, lean:fat gain ratio, feed consumption, and/or feed efficiency greater than or equal to the growth rate, weight gain, lean muscle gain, lean:fat gain ratio, feed consumption, and/or feed efficiency of an animal that has received the growth promotant but has not received Composition I. In some embodiments, the growth rate, weight gain, lean muscle gain, lean:fat gain ratio, feed consumption, and/or feed efficiency of the animal is increased at least 1%, at least 2%, at least 3%, at least 5%, at least 10%, or at least 15%, such as from 1-25%, from 2-20%, from 3-15%, or from 5-10% compared to an animal that has received the growth promotant but has not received Composition I.

Meat obtained from the animal also may have a quality greater than or equal to the quality of meat obtained from an animal that has received a growth promotant but has not received Composition I. Meat quality may be assessed on the basis of carcass maturity, firmness, texture, color of the lean, amount of subcutaneous fat, and/or amount and distribution of marbling (i.e., intramuscular fat) within the lean. Meat quality also may be assessed subjectively on the basis of tenderness, juiciness, and flavor. Accordingly, an animal that has been co-administered Composition I and the growth promotant may have a quality value at harvest greater than or equal to the value of an animal that has received the growth promotant but has not received Composition I. The animal's quality value may be increased by at least 1%, at least 2%, at least 3%, at least 5%, at least 10%, at least 15%, or at least 20%, such as from 1-25%, from 1-20%, from 2-15%, from 2-10%, or from 1-5% compared to the value of an animal that has received the growth promotant but has not received Composition I.

In some embodiments, co-administration of Composition I and a β-agonist may result in elevated interim body weight, increased yield grade, increased marbling score, increased 12^{th} rib fat, increased lean muscle area, and/or increased intramuscular fat.

In some embodiments, Composition I and a growth promotant are co-administered to an animal for a period of time. For example, Composition I and the growth promotant may be co-administered for 1-60 days, such as for 1-30 days, 1-20 days, 5-20 days, 10-60 days, 10-40 days, 10-20 days, 20-40 days, or 25-45 days, prior to harvesting the animal. Composition I may be administered for a period of time prior to administering the growth promotant, during growth promotant administration, after growth promotant administration, or any combination thereof. In some embodiments, the β-agonist is fed to cattle from 1-60 days prior to harvest. In other embodiments, the β-agonist is fed to turkeys for the last 14 days prior to harvest, or from 7-14 days prior to harvest. In certain other embodiments, the β-agonist is administered for a period of time sufficient to result in a desired weight gain in the animal, such as from 10-150 pounds, from 30-120 pounds, or from 45-90 pounds.

In certain embodiments, ractopamine hydrochloride is fed to cattle from 28 to 42 days prior to harvest. In other embodiments, ractopamine is fed to turkeys for the last 14 days prior to harvest, or from 7 to 14 days prior to harvest.

In certain other embodiments, zilpaterol hydrochloride is fed to cattle for 20 days prior to harvest. Zilpaterol administration typically is discontinued for a withdrawal period of at least 3 days prior to harvesting the animal, such as for 3-10 days prior to harvesting the animal. Administration of Composition I may continue during the withdrawal period.

Composition I and a growth promotant may be administered simultaneously or substantially simultaneously to the animal on a daily basis for an effective period of time, e.g., throughout the period during which the growth promotant is administered. When administered simultaneously, Composition I and the growth promotant may be administered as separate compositions simultaneously or sequentially, as a combination (e.g., Composition II), or either or both may be admixed with an animal feedstuff. For example, Composition I component(s) and the growth promotant may be combined with the animal's feed or water and administered simultaneously to the animal. In another example, a pharmaceutical composition comprising (i) the growth promotant and (ii) Composition I may be formed and administered to the animal.

Alternatively, Composition I and a growth promotant need not be administered simultaneously. There are multiple methods of sequential administration. For example, Composition I, or one or more components of Composition I, may be administered followed, or preceded, substantially immediately (e.g., within several minutes to an hour) by the growth promotant. In another example, the animal is administered the growth promotant at a first time, and is administered Composition I, or one or more components of Composition I, at a subsequent time during the day. Alternatively, the animal is administered Composition I, or one or more components of Composition I, at a first time, and is administered the growth promotant at a subsequent time. The first and subsequent times may be, e.g., a first feeding period of a day and a subsequent feeding period of the day.

Composition I may be administered to the animal for a period of time prior to administering a growth promotant to the animal. For example, Composition I may be administered to the animal for at least 10 days, at least 20 days, or at least 30 days, such as for 10-90 days, 30-60 days or 30-45 days, prior to initiating the growth promotant administration. In one embodiment, administration of Composition I continues while the growth promotant is administered to the animal. Thus, Composition I and the growth promotant are co-administered for a period of time as previously discussed.

In some embodiments, one of Composition I and a growth promotant is administered for a first effective period and the other is administered for a second effective period, such that the first effective period and the second effective period overlap. In another embodiment, administration of Composition I is discontinued prior to administering the growth promotant to the animal, leading to a time interval between suspension of Composition I administration and initiation of the growth promotant administration. The beneficial effects of Composition I persist beyond discontinuation of Composition I administration. Thus, the interval between suspension of Composition I administration and initiation of the growth promotant administration may be up to 60 days, such as from 1 day to 60 days, from 1 day to 45 days, from 1 day to 30 days, from 1 day to 20 days, from 1 day to 15 days, or from 1 day to 10 days. Composition I may be administered regularly (e.g., daily) for a period of time, such as for at least 1 day, at least 3 days, at least 5 days, at least 7 days, at least 14 days, at least 30 days, or at least 60 days before suspension. Following suspension of Composition I administration for a selected time interval, the growth promotant may be administered regularly (e.g., daily) for a subsequent period of time, such as for up to 20 days or up to 40 days.

### XI. EXAMPLES

The following examples are provided to illustrate certain effects of Composition I on animals' immune function and/or animals' response to a stressor, such as pregnancy or heat stress. A person of ordinary skill in the art will appreciate that the scope of the disclosed embodiments is not limited to the features exemplified by these working embodiments.

### Example 1

An experiment was conducted with sheep with the goal of determining the ability of Composition I to increase expression of neutrophil L-selectin, a marker of the innate immune system, in immunosuppressed animals. Animals (six per group) were divided into two groups: Control and Experimental. The Control group received a high energy ration consisting of chopped hay available ad libitum, one pound of ground corn per head per day and one pound of baked wheat mill run per head per day for a period of 28 days. During this time, they also received twice daily injections of dexamethasone, an immunosuppressive drug. The Experimental group received daily intake of Composition I (5 grams per head per day) for 28 days and received the same diet and dexamethasone injection protocol as the Control. This composition of the Experimental group was 65.8 weight percent of mineral clay, 0.20 weight percent of endoglucanohydrolase, 9.0 weight percent of glucans and glucomannan, and 25 weight percent of calcined diatomaceous earth. At the end of the study, blood samples were recovered and neutrophils were purified using Percoll gradient centrifugation. The amounts of L-selectin expression in neutrophils were assessed using Western blotting techniques and antibodies specific for L-selectin.

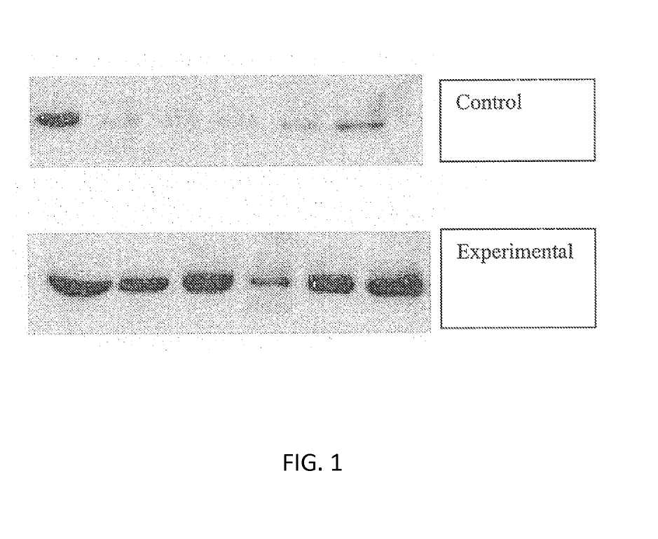

As shown in FIG. 1, top panel, animals that did not receive Composition I had low and variable expression of L-selectin. As shown in FIG. 1, lower panel, animals that received Composition I demonstrated a consistent increase in L-selectin expression. The top panel represents six Control, immunosuppressed animals. The lower panel represents six Experimental immunosuppressed animals which received Composition I in their diet.

### Example 2

In this study, stimulation of the innate immune system in sheep was examined when the Experimental composition of Example 1 was provided in a pelleted diet. The basal diet consisted of 21.55% barley, 10.0% canola meal, 5% distillers grains, 40% ground corn, 1.50% limestone, 0.01% manganese sulfate, 0.01% microvitamin E, 4.0% molasses, 0.25% mono-cal, 0.25% potassium chloride, 0.60% sodium chloride, 0.03% sodium selenite, 15.79% wheat mill run, 0.01% zinc sulfate, 0.75% ammonium sulfate and 0.2 5% cobalt sulfate. When the Experimental composition was added to this diet, it was included at 0.6% replacing that portion of wheat mill run. Twenty-eight sheep were assigned to four treatments which consisted of a Control group, a group which received the Experimental composition in powdered form, a group which received the Experimental composition in pelleted form where pellets were formed at a temperature of 160° F., and a group which received the Experimental composition in pelleted form where pellets were formed at 180° F. All animals were immunosuppressed via daily injection of Dexamethasone.

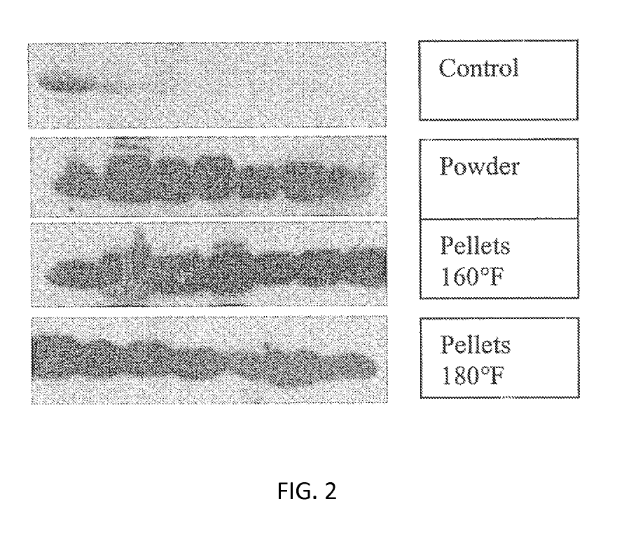

The study was conducted using methods identical to Example 1 except Composition I was administered in pellets that were manufactured by forming the pellets at high temperatures. The rationale for conducting this study was to determine whether heating of Composition I (as is required in pellet formation) might inactivate the ability of Composition I to augment innate immunity. As shown in FIG. 2, sheep (Control) which did not receive Composition I expressed very low levels of L-selectin in neutrophils. The provision of the Experimental composition even in a pelleted (heated) form still increased expression of neutrophil L-selectin markedly.

In FIG. 2, the uppermost panel represents neutrophil L-selectin expression in immunosuppressed animals fed a control diet without Composition I. The second panel (Powder) represents L-selectin expression in immunosuppressed animals which received the Experimental composition in unheated freely-mixed form as in Example 1 (Experimental group). Panels 3 and 4 represent neutrophil L-selectin expression in immunosuppressed animals which received the Experimental composition in pelleted forms. The pellets used in Panel 3 were formed by heating to 160° F. and Panel 4 pellets were heating to 180° F. during manufacture of the feeds.

### Example 3

An experiment was performed with rats to investigate whether Composition I had ability to augment innate immunity in a non-ruminant model. In this study, rats were assigned to one of two treatments: a Control group (un-supplemented diet) and an Experimental group where Composition I of Example 1 was added to the diet at 1% of dry weight of feed. In this experiment, rats were fed a commercial ground rat chow with or without the Experimental composition. Immunosuppression using dexamethasone injection protocols were not utilized in this study. Following 14 days, blood samples were taken from anesthetized rats via cardiac puncture. Neutrophils were isolated from blood samples using Percoll gradient centrifugation and total RNA was isolated using TriZol®.

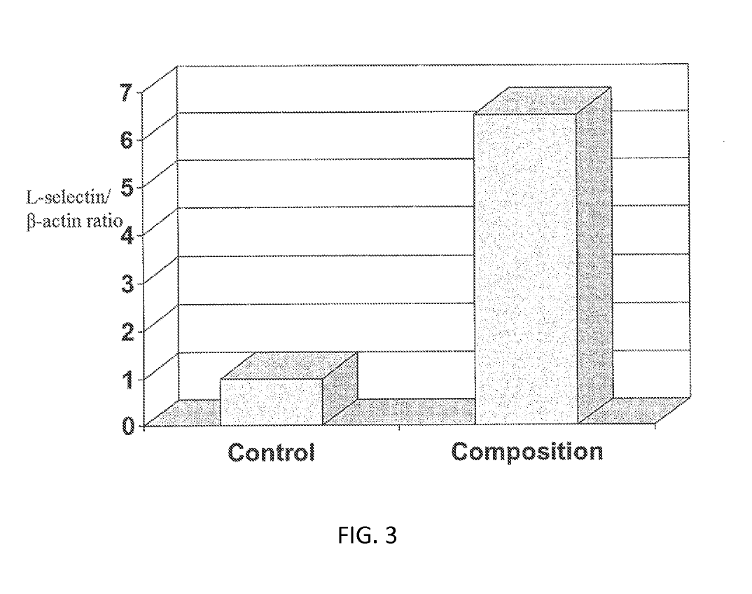

The concentration of the messenger RNA (mRNA) encoding rat L-selectin in the neutrophil RNA samples was then determined by quantitative reverse transcriptase polymerase chain reaction (QRT-PCR) using primers which were specifically developed for assay of rat L-selectin. The amounts of L-selectin mRNA were standardized by showing them as a proportion of β-actin mRNA, which is expressed in all cells at a fairly constant level. As shown in FIG. 3, and in agreement with the results in Examples 1 and 2, Composition I increased expression of L-selectin mRNA by greater than 6-fold (P<0.05).

This study demonstrated that the increased expression of L-selectin protein as shown in by Western blotting in Examples 1 and 2 may be caused by an increase in the mRNA encoding this protein. This implies that Composition I alters the rate of transcription of the gene encoding L-selectin.

### Example 4

Neutrophils, cells of the innate immune system, are able to signal and thereby up-regulate the production of antibodies by the acquired immune system through the secretion of interleukin-1β (IL-1β). To investigate the ability of Composition I to induce neutrophils to increase synthesis of IL-1β, the concentration was assessed of IL-1β in neutrophils taken from the same sheep as described in Example 1. To complete this study, Western blotting and antibodies specific for IL-1β were used.

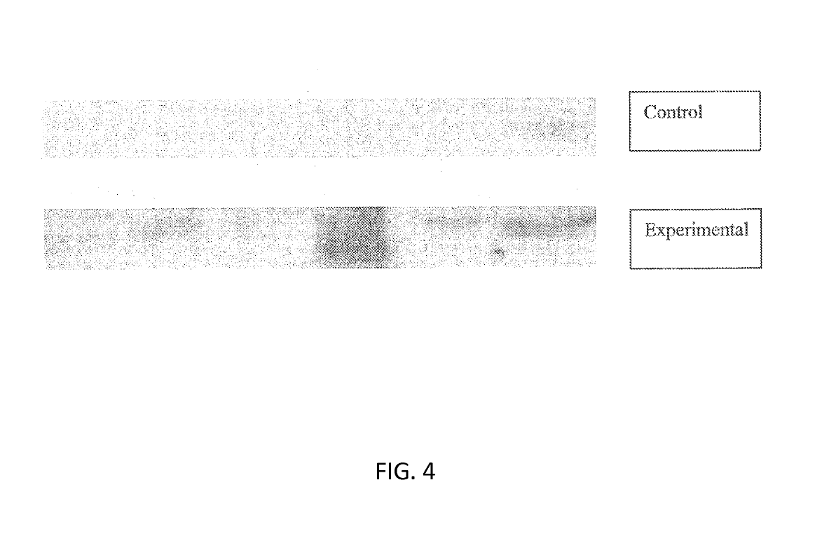

As shown in FIG. 4, animals which did not receive daily provision of Composition I contained virtually undetectable levels of IL-1β; however, provision of Composition I to animals caused a marked increase in the expression of IL-1β (P<0.05). In FIG. 4, the top panel represents six Control-fed immunosuppressed animals. The lower panel represents six Experimental composition-fed immunosuppressed animals which received Composition I. Concentrations of IL-1β were determined using Western blot analysis and an antibody specific for IL-1β.

These data indicate that Composition I not only increases markers of innate immunity (e.g., L-selectin; Examples 1, 2 and 3) but also increases expression of the key signaling molecule (i.e., IL-1β) that up-regulates the adaptive immune system.

### Example 5

The goal of this experiment was to determine which genes were differentially-expressed in neutrophils after feeding Composition I to peri-parturient dairy cattle. In this study, the mechanism(s) by which Composition I increased the expression of IL-1β in neutrophils was examined. Peri-parturient dairy cattle are a good model because the stress of pregnancy leads to immunosuppression, making the cows particularly susceptible to infection.

In this experiment, eight peri-parturient dairy cattle were assigned to a Control diet that did not have the Experimental composition and eight cattle were assigned to an Experimental group that received an embodiment of Composition I in their diet (56 grams per day per head). Animals were fed the diets for approximately 28 days until parturition. At 12-15 hours following parturition, 500 ml samples of blood were recovered via jugular puncture and neutrophils were prepared via large-scale Percoll gradient centrifugation.

RNA was isolated from neutrophils using the TriZol® method and then reverse-transcribed into cDNA using reverse transcriptase. During reverse transcription, differently-colored nucleotide-based dyes (Cy3 and Cy5) were employed such that complementary DNAs (cDNAs) synthesized from the two different treatment (Control and Experimental) groups incorporated different colors. The cDNA samples from Experimental and Control groups were then applied to a BoTL-5 microarray slide. This microarray was prepared at the Center for Animal Functional Genomics at Michigan State University and contains 1500 genes (each arrayed in triplicate) upon a glass slide. The cDNAs generated from the Experimental and Control group samples were then allowed to compete for binding to the 1500 genes on the array and the relative expression of the genes was then assessed by comparing relative abundance of Cy3 and Cy5 signals on each spot on the array. Data were then statistically analyzed to identify those genes which were differentially-expressed (those genes where P<0.05).

The results showed that greater than 20 genes were differentially expressed (P<0.05) in bovine neutrophils taken from the Experimental group. Interleukin-converting enzyme (ICE) was one such up-regulated gene. This was confirmed using QRT-PCR and primers specific to the bovine ICE sequence. ICE is the rate-limiting enzyme in the conversion of inactive pro-IL-1β to the active, secreted IL-1β. Thus, Composition I may up-regulate adaptive immunity (i.e., such as increasing antibody titer) through its ability to increase expression of neutrophil ICE activity and, consequently, secretion of IL-1β.

### Example 6

A total of 60 cows on a commercial dairy were balanced for DIM, parity and milk production and assigned to 1 of 2 treatment groups fed (1) an embodiment of Composition I comprising between 15% and 40% silica, between 50% and 81% mineral clay, between 1.0% and 5.0% β-glucans, between 0.05% and 3.0% β-1,3 (4)-endoglucanohydrolase and between 1% and 8.0% mannan (EX, 30 cows) or (2) control (CON, 30 cows) diets for 52 days post calving. At 52 days of lactation cows were randomly selected (n=12) from both groups (6 EX and 6 CON) and housed in environmentally controlled modules for 21 days. Composition I was top-dressed 2×/day with molasses as the carrier and the CON cows received the molasses carrier 2×/day. Both were mixed into the top one-third of the TMR. During the environmental room phase of the study cows fed Composition I (EX) had higher feed intake than CON during heat stress (HS) (46.8 kg vs. 42.9 kg, P<0.0001) and no difference during thermoneutral (TN). A temperature-humidity index (THI) threshold of 68 or greater was used to achieve HS. Feeding Composition I maintained a numerical 1 kg milk yield advantage compared with CON (30.3 kg vs. 31.4 kg, P=0.26) during HS but not during TN. Cows fed Composition I had lower milk fat (%) (4.2% vs. 3.8%, P=0.02) and milk protein (%) (P=0.04). There was no difference in 3.5% FCM between treatments. Water consumption was lower (12.4 liter/day in Composition I treated cows, P<0.01) than control cows. Respiration rates were lower in treated cows at 1400 hours and 1700 hours (4.7 and 8.4 less respirations/minute, P=0.05, <0.001) and rectal temperatures were also lower (0.15° Celsius and 0.25° Celsius lower that CON, P=0.05, <0.001) in treated cows. Feeding Composition I reduced physiological responses to heat stress in lactating dairy cows.

### Example 7

A total of 30 cows on a commercial dairy were balanced for DIM, parity and milk production and assigned to 1 of 2 treatment groups fed Composition I (EX, 15 cows) or control (CON, 15 cows) diets for 90 days post calving. At 90 days of lactation, cows were randomly selected (n=12) from both groups (6 EX and 6 CON) and housed in environmentally controlled modules for 21 days. Composition I was top-dressed 2×/day with molasses as the carrier. The CON cows received the molasses carrier 2×/day. Both were mixed into the top one-third of the TMR. During the environmental room phase of the study, cows fed Composition I (EX) had higher feed intake than CON during heat stress (HS) (46.8 kg vs. 42.9 kg, P<0.0001) and no difference during thermoneutral (TN). A temperature-humidity index (THI) threshold of 68 or greater was used to achieve HS. Feeding Composition I maintained a numerical 1 kg milk yield advantage compared with CON (30.3 kg vs. 31.4 kg, P=0.26) during HS but not during TN. Cows fed Composition I had lower milk fat (%) (4.2% vs. 3.8%, P=0.02) and milk protein (%) (P=0.04). There was no difference in 3.5% FCM between treatments. Water consumption was lower (12.4 liter/day in Composition I treated cows, P<0.01) than control cows. Respiration rates were lower in treated cows at 1400 hours and 1700 hours (4.7 and 8.4 less respirations/minute, P=0.05, <0.001) and rectal temperatures were also lower (0.15° Celsius and 0.25° Celsius lower that CON, P=0.05, <0.001) in treated cows. Feeding Composition I reduced physiological responses to heat stress in lactating dairy cows.

Experimental Design:

The study consisted of two phases; 1) the commercial dairy, and 2) the controlled environmental chambers. During the commercial dairy phase, multiparous lactating Holstein cows (n=30) were balanced by DIM, milk production and parity (91±5.9 DIM, 36.2±2.5 kg/day, and 3.1±1.4). Cows were separated into one of two groups. The control group received the base TMR with no supplement. The treatment group was fed the base diet plus 56 grams/head/day of Composition I (EX) mixed into the TMR. Daily milk production was measured. The dairy phase lasted for 45 days. The dairy portion was used to meet the manufacture's recommended 45 days feeding for EX to function.

After the on-dairy portion was complete, 12 cows (6 control and 6 treatment) were housed in environmentally controlled rooms. Cows continued the ARC portion in the same treatment groups from the on-dairy portion.

The ARC portion lasted for 21 days. Cows were subjected to 7 days of TN conditions, 10 days of HS, and 4 days of recovery (TN). Feed intake, milk production, and milk composition were measured daily. Rectal temperatures and respiration rates were recorded 3×/day (600, 1400, and 1800 hours). Blood samples were taken on days 7 (TN), 8 (HS), 10 (HS), 17 (HS) and 18 (TN) during the ARC segment.

Statistical analyses were performed using the PROC MIXED procedure (version 9.3, SAS Institute, Cary, N.C.). Cow was the experimental unit (ARC portion). Data is presented in least square means with significance declared with a P-value ≦0.05. (See Table 1, below).

Feeding the disclosed composition to heat stressed dairy cows maintained feed intake during heat stress. Milk yield had a numeric (1 kg) advantage with Composition I treatment but did not differ significantly. Respiration rate and rectal temperatures were lower in treated animals during heat stress. There was also a reduction in SCC with treatment. Serum cortisol levels were lower in on 8 days (the first day of heat stress) at 2000 hours in Composition I supplemented cows (P=0.03).

TABLE 1

| | Control | | | Composition I (EX) | | | | |
|---|---|---|---|---|---|---|---|---|
| Item | TN | HS | Recovery | TN | HS | Recovery | SEM | P-value |
| Feed intake (kg) | 46.1 | 42.9 | 47.5 | 47.1 | 46.8* | 49.1 | 1.04 | 0.01 |
| Milk yield (kg) | 33.1 | 30.3 | 30.4 | 33.9 | 31.4 | 31.3 | 1.02 | 0.23 |
| Resp/min | | | | | | | | |
| 600 | 26.9 | 31.9 | 28.3 | 26.6 | 30.4 | 27.9 | 1.40 | 0.40 |
| 1400 | 34.3 | 63.1* | 35.3 | 30.1 | 58.3 | 35.5 | 2.99 | 0.20 |
| 1800 | 34.9* | 60.8* | 32.1 | 29.5 | 52.4 | 29.7 | 2.62 | 0.01 |
| Rectal Temp (° C.) | | | | | | | | |
| 600 | 38.2 | 38.0 | 37.9 | 38.2 | 38.1 | 38.1 | 0.05 | 0.26 |
| 1400 | 38.0 | 38.7* | 38.0 | 38.1 | 38.5 | 38.1 | 0.09 | 0.77 |
| 1800 | 38.2 | 39.1* | 38.2 | 38.2 | 38.8 | 38.3 | 0.08 | 0.25 |
| FCM (kg/d) | 35.0 | 33.7 | 33.7 | 34.7 | 32.8 | 32.6 | 1.45 | 0.39 |
| Protein (%) | 2.95 | 2.98* | 2.86 | 2.95 | 2.86 | 2.79 | 0.07 | 0.15 |
| Protein (kg) | 0.94 | 0.89 | 0.87 | 0.97 | 0.89 | 0.86 | 0.3 | 0.13 |
| Lactose (%) | 4.87 | 4.85 | 4.99 | 4.89 | 4.78 | 4.96 | 0.08 | 0.61 |
| SCC | 20.3 | 23.9 | 59.4* | 19.6 | 22.9 | 26.3 | 9.12 | 0.03 |

### Example 8

Thirty six male CD rats (ca. 225 grams) were randomly assigned to six treatment groups for a feeding trial. Animals were fed one of the following six diets ad libitum for 28 days:

A. Control diet (Teklad 8604 powdered diet).

B. Diet supplemented with yeast cell wall preparation (including β-glucans and glucomannan).

C. Diet supplemented with diatomaceous earth and β-1,3(4)-endoglucanohydrolase.

D. Diet supplemented with the yeast cell wall preparation of Composition B, diatomaceous earth, and β-1,3(4)-endoglucanohydrolase.

E. Diet supplemented with the yeast cell wall preparation of Composition B, diatomaceous earth, β-1,3(4)-endoglucanohydrolase, and mineral clay.

F. Diet supplemented with 0.5% w/w of a commercially available supplement, comprising 9 wt % Safmannan® yeast cell wall material (source of β-glucans and mannans), 25 wt % diatomaceous earth, 0.02 wt % Trichoderma extract (a source of β-1,3(4)-endoglucanohydrolase), 65.98 wt % AB20™ bentonite, and a mixture of B-vitamins.

The amounts of yeast cell wall extract, diatomaceous earth, β-1,3(4)-endoglucanohydrolase and mineral clay used to supplement the diet in Compositions B-E were selected to reflect the amounts that would be added if the diet were supplemented with the commercial supplement recited in Composition F.

On day 28, rats were anesthetized with a mixture of ketamine and xylazine and blood samples (6-10 mL) were taken via cardiac puncture. Neutrophils were isolated from blood samples via Percoll gradient centrifugation. RNA was isolated from a portion of neutrophils in all animals using the Trizol® method. This was then used to quantify concentrations of L-selectin, interleukin-8 receptor (IL-8R) and β-actin mRNAs. Another portion of neutrophils from all animals were used in a phagocytosis (cell killing assay). In this assay, neutrophils isolated from rats were combined with Staphylococcus aureus in a ratio of 30:1 S. aureus bacteria to neutrophil. Neutrophils were allowed to “react” with bacteria for 3 hours after which S. aureus viability was assessed spectrophotometrically.

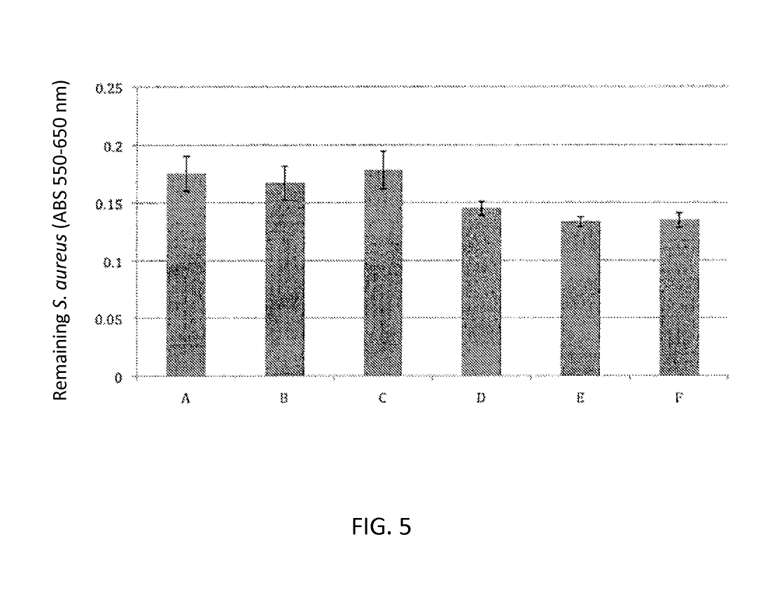

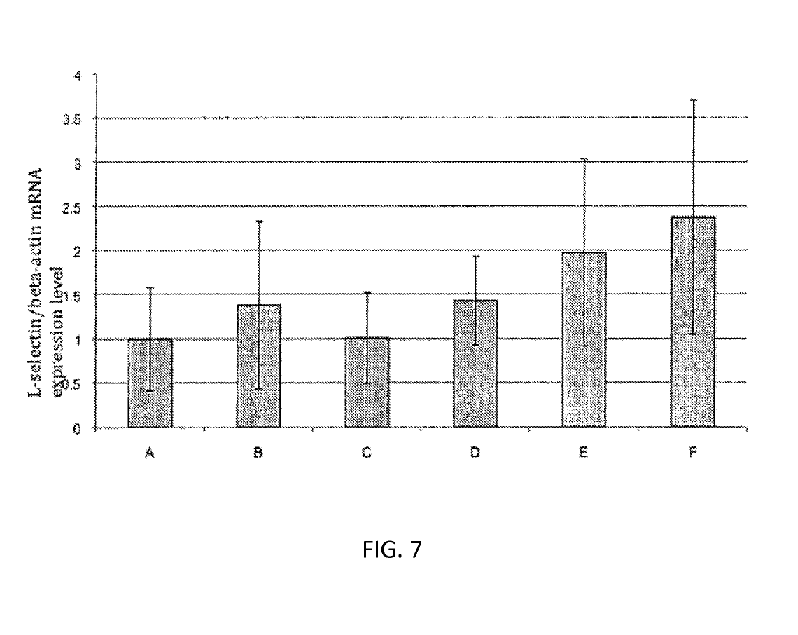

The study found that Composition B (yeast cell wall preparation) and Composition C (diatomaceous earth and β-1,3(4)-endoglucanohydrolase) had no significant effect on any of the three tested markers of innate immunity, as compared to Composition A (control diet) (FIGS. 5-7).

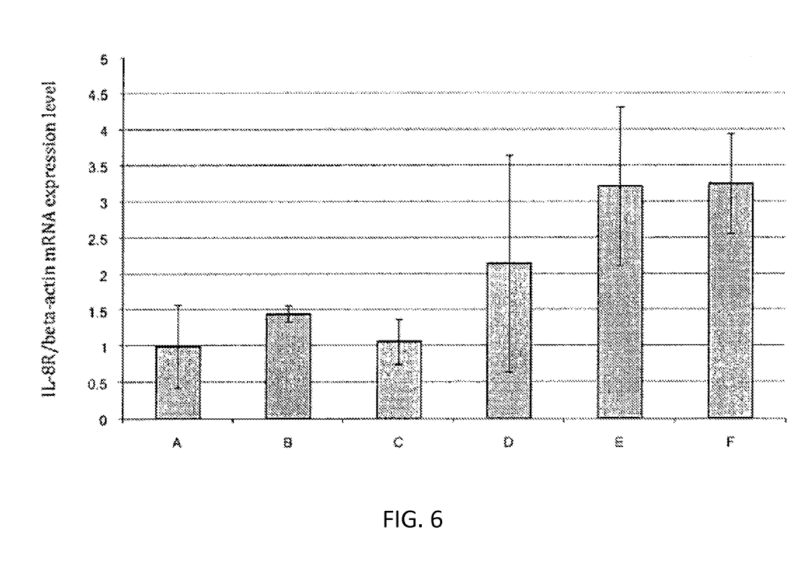

While neither Composition B nor Composition C produced a significant effect on the ability of neutrophils to phagocytose S. aureus, Composition D, which represents a combination of Compositions B and C, unexpectedly improved phagocytosis by 20%, which is significant (FIG. 5). Furthermore, the addition of a mineral clay (Composition E) resulted in a significant improvement in the IL-8R marker, as compared to the control (Composition A) (FIG. 6). Composition E also caused a further significant reduction in S. aureus viability as compared to Composition D (FIG. 5). Composition F (commercially available supplement) was found to have the ability to regulate the three measured markers in innate immunity and substantially mimicked the results obtained with Composition E, indicating that the B vitamins included in the commercially available supplement do not significantly affect regulation of these markers of innate immunity (FIGS. 5-7).

### Example 9

A study was conducted to identify genes expressed by circulating immune cells that are regulated by a commercial embodiment of Composition I. Rats (n=6 per group) were randomly assigned to Composition I and control groups. Composition I was supplemented in the diet at 0.5% in the Composition I group. Total RNA was purified from whole blood and gene expression was analyzed with the use of the Rat Innate and Adaptive Immune Responses RT2 Profiler Polymerase Chain Reaction (PCR) Array (SABiosciences, Qiagen). A total of 84 target genes were present on the array. Gene expression of circulating immune cells was analyzed at seven, fourteen, twenty-one and twenty-eight days of Composition I supplementation. The expression of 67 genes changed following Composition I supplementation across the time points. Table 2 lists the genes with altered gene expression following Composition I supplementation and includes information indicating stimulation (+) or repression (−) of gene expression.

TABLE 2

| Gene | Repressed | Gene | Induced | Reference Genes |
|---|---|---|---|---|
| Crp | − | Ifnb1 | + | Actb |
| Mbl2 | − | Cd80 | + | Ldha |
| Apcs | − | Tlr1 | + | Rplp1 |
| Il5 | − | Tlr6 | + | |
| Ifna1 | − | Mapk8 | + | |
| Ccl12 | − | Nod2 | + | |
| Csf2 | − | Ccr8 | + | |
| Il13 | − | Irak1 | + | |
| Il10 | − | Cd1d1 | + | |
| Gata3 | − | Stat4 | + | |
| Stat3 | − | Il1r1 | + | |
| C3 | − | Faslg | + | |
| Tlr3 | − | Irf3 | + | |
| Ccl5 | − | Ifnar1 | + | |
| Mx2 | − | Slc11a1 | + | |
| Nfkb1 | − | Tlr4 | + | |
| Nfkbia | − | Cd86 | + | |
| Tlr9 | − | Casp1 | + | |
| Cxcl10 | − | Ccr5 | + | |
| Cd4 | − | Icam1 | + | |
| Il6 | − | Camp | + | |
| Ccl3 | − | Tlr7 | + | |
| Ccr6 | − | Irf7 | + | |
| Cd40 | − | Rorc | + | |
| Ddx58 | − | Cd40lg | + | |
| Il18 | − | Tbx21 | + | |
| Jun | − | Casp8 | + | |
| Tnf | − | Il23a | + | |
| Traf6 | − | Cd14 | + | |
| Stat1 | − | Cd8a | + | |
| | | Cxcr3 | + | |
| | | Foxp3 | + | |
| | | Lbp | + | |
| | | Mapk1 | + | |
| | | Myd88 | + | |
| | | Stat6 | + | |
| | | Agrin | + | |
| | | IL33 | + | |

Additional subject matter concerning Composition I is found in U.S. Pat. No. 7,939,066, U.S. Pat. No. 8,142,798, U.S. Pat. No. 8,236,303, U.S. Pat. No. 8,431,133, U.S. Pat. No. 8,568,715, U.S. Provisional Application No. 61/856,544, and U.S. Provisional Application No. 61/859,689, each of which is incorporated herein by reference in its entirety.

### Example 10

An experiment is performed with thirty Angus steers weighing approximately 500 kg. Animals are randomly divided into two groups and fed individually. Group 1 receives a standard finishing ration. Animals in Group 2 receive supplementation of Composition I beginning on Day 0 of the study. The dose of Composition I is 50 g/head/day. At 6 weeks prior to slaughter, rations for animals in both groups are supplemented with ractopamine-HCl (OptiFlex; 250 mg/head/day) until day of slaughter. On days 42, 28, 14 and 0 prior to slaughter, physiological assessments of animal health and performance are made. These assessments included body temperature, respiration rate, heart rate, lameness, stiffness, muscle tremors, blood creatinine and blood glucose. After slaughter evidence of muscle damage, kidney damage, kidney weight and heart weights are assessed. Several carcass traits are determined. These included marbling, rib eye area, kidney, pelvic and heart fat (KPH), back fat thickness and hot carcass weight. Performance characteristics, including average daily fain, feed intake and feed/gain (feed efficiency) are also determined. At time of slaughter, blood samples are taken and neutrophils are isolated for assay of immunological competence.

Expected Results:

No differences are expected in animal performance in Group 1 versus Group 2; however, it is predicted that indicators of stress (elevated body temperature, elevated respiration, elevated heart rate, tissue damage [kidney and heart], reduced kidney weight and increased heart rate]), will be ameliorated in Group 2-fed animals (P<0.05) compared to Group 1-fed animals. Biological markers of immune function (neutrophil L-selectin and interleukin-1-beta) will be elevated in the Composition 1-fed animals.

### Example 11

An experiment is performed with sixty Angus steers weighing approximately 500 kg. The study is completed as a 2×2 factorial design. Animals are randomly divided into four groups and fed individually. The four treatments are shown in the following Table:

| | Administration of | Administration of |
|---|---|---|
| Group | Zilpaterol-HCl | Composition 1 |
| 1 | None | None |
| 2 | None | Duration of study |
| 3 | Last 20 days of | None |
| | finishing period | |
| 4 | Last 20 days of | Duration of study |
| | finishing period | |

At 6 weeks prior to slaughter, rations for animals in Groups 3 and 4 are supplemented with zilpaterol-HCl (Zilmax: 6.8 g/ton) until day of slaughter. On days 42, 28, 14 and 0 prior to slaughter, physiological assessments of animal health and performance are made. These assessments include body temperature, respiration rate, heart rate, lameness, stiffness, muscle tremors, blood creatinine and blood glucose. After slaughter evidence of muscle damage, kidney damage, kidney weight and heart weights are assessed. Several carcass traits are determined. These included marbling, rib eye area, kidney, pelvic and heart fat (KPH), back fat thickness and hot carcass weight. Performance characteristics, including average daily fain, feed intake and feed/gain (feed efficiency) are also determined. At time of slaughter, blood samples are taken and neutrophils are isolated for assay of immunological competence.

Expected Results:

Feeding Composition I (Group 2 versus group 1) increases performance traits of beef cattle including an increase in feed intake, an increase in feed efficiency, an increase in weight gain, an increase in lean muscle gain and an increase in lean:fat gain. Composition I also increases markers of immunocompetence in neutrophils (L-selectin and IL-1B mRNAs). Feeding zilpaterol-HCl increases evidence of biological stress in animals (Group 3 versus Group 1). Specifically, zilpaterol-HCl increases body temperature, respiration rate and heart rate and increased objective assays of stiffness, lameness and tremors. Further, zilpaterol-HCl increases blood creatinine concentrations and reduces blood glucose concentrations. Zilpaterol alters absolute weights of organs including liver, kidney and heart. Zilpaterol-HCl increases biological markers of performance including increased muscularity and reduced fat deposition. An interaction will be determined when comparing Group 4-fed animals with Group 3-fed animals. Whereas the addition of zilpaterol-HCl to the ration is expected to cause several deleterious effects in health, these effects are predicted to be mitigated by the inclusion of Composition I in the ration.

### Example 12

Feeding OmniGen-AF® (Composition I or OG; Prince Agri Products, Inc., Quincy, Ill.), a branded proprietary product, at 0.5% of the diet supports immune function in ruminant livestock. Targeted profiling of immune-associated genes in whole blood is an established methodology to evaluate the efficacy of feed additives with immune-altering properties. It was hypothesized that higher daily inclusion rate of OG than 0.5% may be required to optimize immune function. The objective of this study was to evaluate the effect of dietary OG inclusion rate (1% vs. 0.5%) on the expression profile of immune-associated genes. Male CD rats (5/treatment) weighing 180-200 grams had ad libitum access to a diet with 0 (control), 0.5 (1×), or 1% (2×) of OmniGen-AF® for 28 days. At the end of the feeding period, whole blood was collected. RNA was purified from whole blood samples and used to generate cDNA that acted as template in the Rat Innate and Adaptive Immune Responses RT² Profiler PCR array (SABiosciences). Using PROC GLM, cDNA abundance of immune-associated genes from control and supplemented groups (0.5 or 1%) were compared, with a P<0.05 cut-off value for significance. Of the 79 immune-associated genes that were expressed above the detection limit in all samples, 16 (7 up-regulated) and 13 genes (8 up-regulated) were altered by 0.5% and 1% OG supplementation, most of which (11 with 6 up-regulated) were altered at both OG inclusion rates. Genes that were up-regulated at both rates include IL13 (0.5%: +3.16, 1%: +3.70 fold-change), IL5 (0.5%: +2.64, 1%: +2.62), Irak1 (0.5%: +2.50, 1%: +1.98), Nod2 (0.5%: +1.83, 1%: +2.02), IFNa1 (0.5%: +1.81, 1%: +2.10), and Cd80 (0.5%: +1.77, 1%: +2.47). Genes that were down-regulated at both inclusion rates include TLR3 (0.5%: −2.22, 1%: −2.39), CxCL10 (0.5%: −2.19, 1%: −2.26), STAT1 (0.5%: −2.07, 1%: −1.99), STAT3 (0.5%: −2.05, 1%: −1.92), and NFκb1 (0.5%: −1.84, 1%: −1.75). In conclusion, the results suggest that OG supplementation inclusion rate independently promotes immune function through various pathways including pathogen recognition, adaptive immune cell activation, and various transcription factors.

### Example 13

OmniGen-AF® (Composition I or OG; Prince Agri Products, Inc., Quincy, Ill.) is a branded proprietary product shown to augment immune function in ruminants and other species. Targeted profiling of immune-associated genes in whole blood is an effective platform for identification of multiple immune response markers to feed additives with immune-altering properties. The objective of this study was to identify multiple immune response markers that are increased by dietary OG throughout a 28-d supplementation period. It was hypothesized that several immune-associated genes in whole blood are consistently up-regulated during a 28-d supplementation period. Fourteen male CD rats weighing 180-200 grams had ad libitum access to a diet containing 0 (control; n=5, only 28 days) or 0.5% OG for 7 (n=4) or 28 days (n=5). Whole blood was collected at the end of the feeding period. RNA was purified from whole blood samples and used to generate cDNA that acted as template in the Rat Innate and Adaptive Immune Responses RT² Profiler PCR array (SABiosciences). Using PROC GLM, cDNA abundance of immune-associated genes from control and supplemented groups (7 or 28 d) were compared, with a P<0.05 cut-off value for significance. Of the 77 immune-associated genes that were expressed above the detection limit in all samples, 6 genes were up-regulated after 7 d of OG supplementation and 4 genes were up-regulated after 28 d of OG supplementation. Three genes were up-regulated after 7 d (Cd80: +2.40; Irak1: +2.25; Nod2: +2.08 fold-change) as well as after 28 d of OG supplementation (Cd80: +1.77; Irak1: +2.50; Nod2: +1.83 fold-change). In conclusion, the results suggest Cd80, Irak1, and Nod2 as immune response markers that are increased by dietary OG throughout a 28-d supplementation period.

### Example 14

### Animal Management

Crossbred steers (n=336; initial BW=309±22 kg) were utilized in a feedlot finishing trial at the University of Nebraska Panhandle Research Feedlot (PHREC) near Scottsbluff, Nebr. in a 3×2 factorial completely randomized block design. The first factor was the duration of OmniGen-AF® (Composition I, Prince Agri Products, Inc.; Quicny, Ill.) supplementation (4 g/45.5 kg BW) being the last 0, 28, or 56 days of the finishing period. The second factor was supplementation of ractopamine hydrochloride (RAC; Elanco Animal Health; Greenfield, Ind.) at 300 mg/steer/day for the last 28 days of finishing (R) or no beta agonist supplementation (N). The above treatment design provided a total of 6 experimental treatments, 3 with a beta agonist (0R,28R, 56R) and 3 without a beta agonist (0N, 28N, 56N).

Steers were purchased from auction markets in Scottsbluff, Nebr. on Nov. 11, 2013 and St. Ogne, S. Dak. Nov. 22, 2013. On arrival to the PHREC, steers were individually identified (panel tag, metal clip), vaccinated with Express 5 (Boehringer Ingelheim; St. Joeseph, Mo.) and Vision 7 Somnus (Merck Animal Health; Summit, N.J.), treated for parasites with Ivomec (Merial Limited; Duluth, Ga.), and branded. Steers were revaccinated with Express 5 when initial ultrasound data was collected 58 days prior to the targeted marketing date of each BW block. Steers were limit fed a diet consisting of 45% ground alfalfa hay, 35% beet pulp, and 20% of wet distillers grains plus solubles (WDGS; DM basis) for 5 days prior to the start of the experiment. Three-day BW measurements were recorded on day −1, 0 and 1 of the experiment, were averaged, and used as the initial BW for the experiment to reduce variation associated with gastrointestinal tract fill (Stock et al., 1983; Watson et al., 2013). Steers were blocked by the initial BW into heavy, medium, and light BW blocks, stratified by BW and assigned randomly within block to pen for a total of 42 pens (8 steers/pen). Pen was then randomly assigned to one of the six treatments described above. Steers were implanted with Revalor®-XS (Merck Animal Health) on day −1. Steers were adapted to a finishing diet via four step-up diets that replaced alfalfa hay with dry-rolled corn. Step-up diets were fed 3, 4, 7, and 7 days; respectively (Table 1), so that by d 22 of the trial steers were on the finishing diet. Steers were fed a finishing diet consisting of 54% DRC, 25% WDGS, 15% corn silage, 6% supplement (DM basis; Table 1) for 146 for the heavy and medium BW blocks and 173 days for the light BW block (Table 1). All steers were fed a supplement provided via micomachine (Model 271 Weigh and Gain Generation 7; Animal Health International, Greely, Colo.) to provide 30 g/ton Rumensin® (Elanco Animal Health; DM basis) and 90 mg/steer daily of Tylan® (Elanco Animal Health). OmniGen-AF® supplementation (4 g/100 lb BW) was administered through topdressing the delivered finishing diet beginning 56 (56R and 56N) or 28 (28R and 28N) days prior to the targeted marketing date of each BW block throughout the rest of the finishing period. The topdress consisted of 50 g OmniGen-AF® and 100 g fine ground corn carrier (DM basis) fed to achieve the 4 g/45.5 kg per steer. Those pens designated to not receive OmniGen-AF® supplementation (0R and 0N) still received a topdress of fine ground corn as a control. Pens designated to treatments that were to receive a beta agonist (0R,28R, and 56R) were supplemented RAC (300 mg/steer/day) via micromachine (Model 271 Weigh and Gain Generation 7; Animal Health International, Greely, Colo.) beginning 28 days prior to the targeted market date of each BW block and lasted throughout the remainder of the finishing period. Steers had ad libitum access to fresh clean water and their respective diets. Steers were fed once daily for the duration of the study. Diet samples were sent to Servi Tech Labs (Hastings, Nebr.) for analysis.

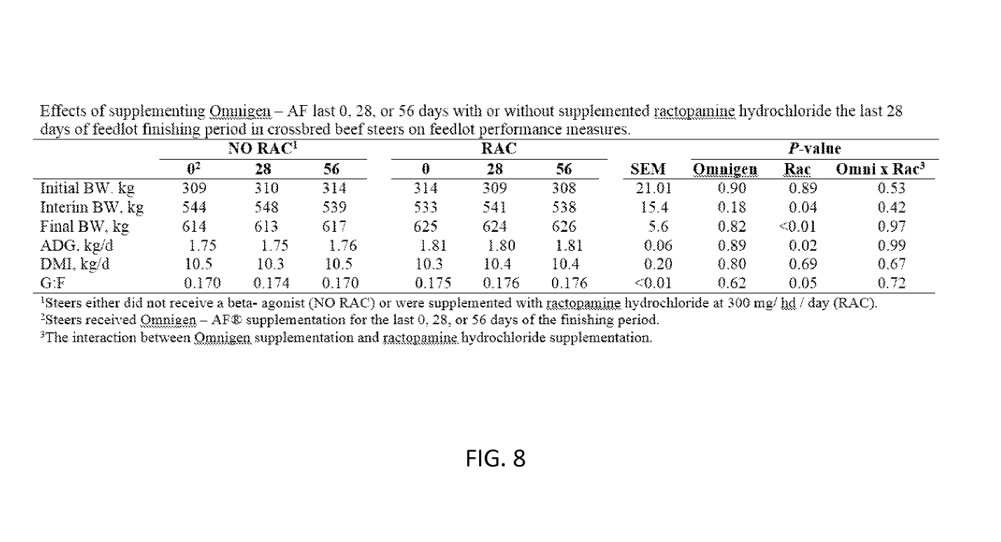

The results are shown in FIG. 8, which illustrates that the interim body weight (BW) in RAC-fed steers was elevated by both 28-day and 56-day OmniGen feeding.

### Ultrasound Data Collection

Ultrasound data measurements of rump fat thickness (RUMP), 12^{th} rib fat thickness (RIB), LM area, and intramuscular fat (IMF) were collected on each steer 58 days prior (initial) to the targeted marketing date of each BW block and then again 2 days prior (final) to steers being harvested. Individual steer BW was also collected at each ultrasound time point. The differences between final and initial ultrasound data were then calculated to determine any body composition change due to treatments imposed.

### Carcass Data Collection and Calculations

Steers in the heavy and medium BW blocks were harvested on day 167 and the light BW block was harvested on day 194. Cattle were not fed the morning prior to being shipped, and were fed ad libitum the day prior to shipment. Carcass data was collected by Diamond T Livestock Services (Yuma, Colo.). Hot carcass weight, and liver scores were recorded the day of harvest. After a 48-hour chill, 12^{th} rib fat depth, LM area, and marbling score (where 300=Slight⁰, 400=Small⁰) were recorded. Carcass adjusted final BW, used in calculation of ADG and G:F, was calculated from HCW using a common dressing percentage of 63% to minimize errors associated with gastrointestinal tract fill. Yield grade was calculated (Boggs and Merkel, 1993) from the equation

Yield Grade=2.50+(6.35×fat thickness,cm)−(2.06×LM area,cm²)+(0.2×KPH,%)+(0.0017×HCW,kg)

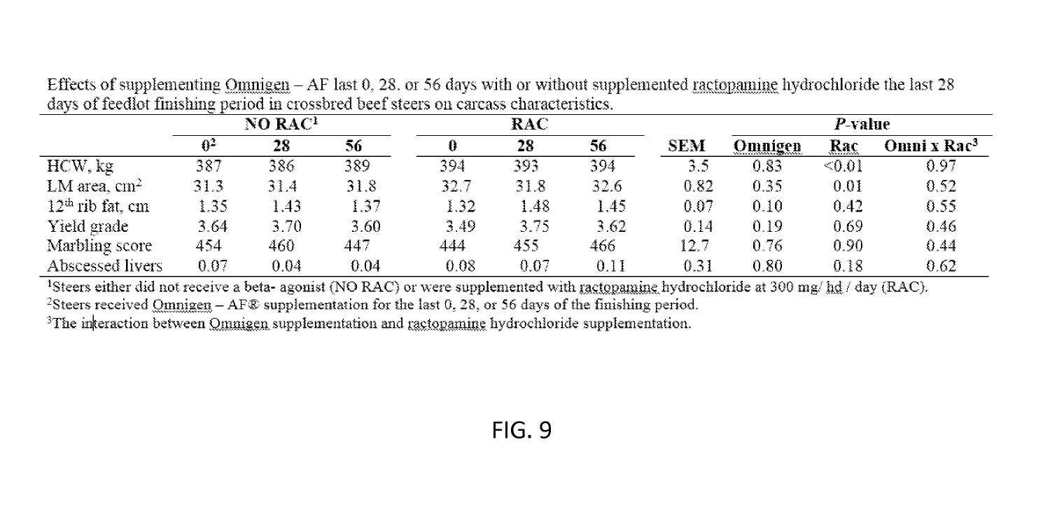

The data provided in FIG. 9 illustrates that the yield grade in the absence of RAC was not affected by OmniGen-AF®; however, in RAC-fed animals, OmniGen-AF® increased yield grade. Also, the Marbling score was not consistently affected by OmniGen-AF® in the absence of RAC, but in RAC-fed steers, OmniGen-AF® increased marbling score in a dose-dependent manner. Furthermore, the 12th rib fat was increased more by OmniGen-AF® in RAC-fed steers than in animals not receiving RAC.

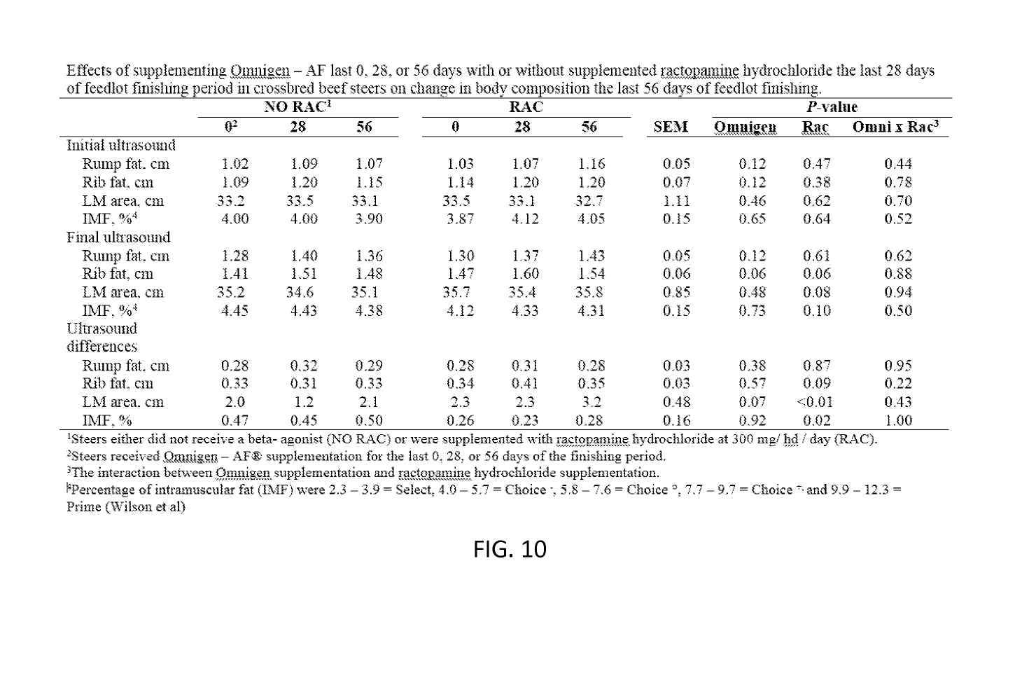

The data in FIG. 10 illustrates that in the absence of RAC, OmniGen-AF® caused a small decrease in the percentage of intramuscular fat (IMF %). However, in the presence of RAC, it increased IMF % (a desirable result). Also, OmniGen increased lean muscle (LM) area (a desirable trait) in RAC-fed animals but not in control-fed animals.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method, comprising administering to an animal (i) a growth promotant, and (ii) Composition I, wherein the Composition I comprises silica, mineral clay, mannans, or any combination thereof.

2. The method of claim 1, further comprising:
identifying an animal to which a growth promotant will be administered or to which at least one dose of a growth promotant has been administered.

3. The method of claim 1, wherein the Composition I further comprises glucan, β-1,3 (4)-endoglucanohydrolase, or both.

4. The method of claim 1, wherein the growth promotant comprises an antibiotic, steroid, hormone, β-agonist, or combination thereof.

5. The method of claim 1, wherein the growth promotant comprises monensin, virginiamycin, tylosin phosphate, melengestrol acetate, ractopamine, zilpaterol or any combination thereof.

6. The method claim 1 where the animal is a mammal, an avian species, a fish, a reptile, or a crustacean.

7. The method of claim 6 where the animal is a bovine, swine, turkey or chicken.

8. The method of claim 1 wherein the growth promotant is administered to the animal for an effective period of time to (i) increase a feed intake of the animal relative to a feed intake of the animal prior to the growth promotant administration, (ii) increase a feed efficiency of the animal relative to a feed efficiency of the animal prior to growth promotant administration, (iii) produce a greater weight gain of the animal relative to a weight gain of an animal that has not received the growth promotant, (iv) produce a greater lean muscle gain of the animal relative to a lean muscle gain of an animal that has not received the growth promotant, (v) produce an increased ratio of lean:fat gain of the animal compared to an animal that has not received the growth promotant, or (vi) any combination thereof.

9. The method of claim 1 where the Composition I is administered to the animal for an effective period of time to (i) increase a feed intake of the animal relative to a feed intake of an animal that has received the growth promotant but has not received the Composition I, (ii) produce a greater weight gain of the animal relative to a weight gain of an animal that has received the growth promotant but has not received the Composition I, (iii) produce a higher harvest value of the animal relative to a harvest value of an animal that has received the growth promotant but has not received the Composition I, or (iv) any combination thereof.

10. The method of claim 1, wherein administering the growth promotant and Composition I to the animal comprises administering to the animal a combination comprising the growth promotant, Composition I and at least one feedstuff.

11. The method of claim 10, wherein the combination comprises an admixture of the growth promotant in a first feedstuff and an admixture of Composition I in a second feedstuff, where the first feedstuff and the second feedstuff are different.

12. The method of claim 1 where the Composition I and the growth promotant are administered simultaneously or sequentially in any order to the animal.

13. The method of claim 12 where the Composition I and the growth promotant are administered to the animal on a daily basis for 1-60 days.

14. The method of claim 1, further comprising administering the Composition I to the animal for 30-60 days prior to administering the growth promotant to the animal.

15. The method of claim 1 where administering the Composition I to the animal promotes a reduction in at least one deleterious symptom or sign observed or measured in the animal associated with administration of the growth promotant.

16. The method of claim 15 where the at least one deleterious symptom or sign comprises:
lameness, stiffness, muscle tremors, muscle damage, kidney damage, an increase or decrease in blood creatinine, an increase or decrease in blood glucose, an increase or decrease in relative and/or absolute weights of kidneys, heart and/or liver, an increase in signs of injury, or a combination thereof;
an aberrant biomarker, wherein the biomarker is an aberrant immune system biomarker or an aberrant inflammation biomarker;
a stress indicator selected from increased panting, increased time lying down, increased temperature, increased sweating, increased heart rate, increased respiratory rate, respiratory alkalosis, decreased feed intake, increased water consumption, rumen acidosis, metabolic acidosis, dark cutters, poor carcass quality, decreased milk production, decreased immune function, or any combination thereof, compared to an animal that has not been administered growth promotant; or
any combination thereof.

17. The method of claim 1 where a stress hormone level in the animal after administration of the Composition I is lower than a level of the stress hormone in an animal that has been administered the growth promotant but has not been administered the Composition I.

18. The method of claim 1 where the Composition I comprises 1-40 wt % silica, 1-25 wt % glucan and mannans, and 40-92 wt % mineral clay.

19. A composition, comprising:
Composition I, where the Composition I comprises silica, mineral clay, mannans, glucan, β-1,3 (4)-endoglucanohydrolase, or any combination thereof; and
a growth promotant.

20. The composition of claim 19, further comprising a feedstuff comprising a feed ration, water, molasses, a mineral supplement, a protein supplement, a premix, a liquid feed, or any combination thereof.

21. The composition of claim 20, where the composition comprises the Composition I in an amount of from 0.1 to 100 kg per ton of feedstuff and the growth promotant in an amount from 0.001 to 1 kg per ton of feedstuff.

22. The composition of claim 19, wherein the growth promotant comprises an antibiotic, steroid, hormone, β-agonist or combination thereof.

23. The composition of claim 22 wherein the β-agonist is dobutamine, isoproterenol, xamoterol, epinephrine, salbutamol (albuterol), levosalbutamol (levalbuterol), fenoterol, formoterol, metaproterenol, salmeterol, terbutaline, clenbuterol, isoetarine, pirbuterol, procaterol, ritodrine, arbutamine, befunolol, bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol, zinterol, or combinations thereof.

24. A method for making a composition, comprising combining (i) a Composition I, and (ii) a growth promotant to produce the composition, wherein the Composition I comprises silica, mineral clay, mannans, or any combination thereof.
